(19) United States
(12) Patent Application Publication
(10) Pub. No.: US 2019/0383817 A1
(43) Pub. Date: Dec. 19, 2019

(54) PREDICTING RESPONSE TO IMMUNOMODULATORY DRUGS (IMIDS) IN MULTIPLE MYELOMA PATIENTS

(71) Applicants: SKYLINEDX B.V., Rotterdam (NL); ERASMUS UNIVERSITY MEDICAL CENTER ROTTERDAM, Rotterdam (NL)

(72) Inventors: Pieter SONNEVELD, Schiedam (NL); Martinus Hendrikus VAN VLIET, s'-Gravendeel (NL); Cornelia Maria DE BEST, Rijpwetering (NL)

(73) Assignees: SKYLINEDX B.V., Rotterdam (NL); ERASMUS UNIVERSITY MEDICAL CENTER ROTTERDAM, Rotterdam (N.)

(21) Appl. No.: 16/067,235

(22) PCT Filed: Feb. 9, 2017
(86) PCT No.: PCT/NL2017/050077

§ 371 Date: Jun. 29, 2018

(30) Foreign Application Priority Data
Feb. 12, 2016 (EP) 16155477.9

(51) Int. Cl. G01N 33/574; C12Q 1/6886; A61K 31/454

(52) U.S. Cl. CPC G01N 33/57426; G01N 33/57484; C12Q 1/6886; C12Q 2600/106; G01N 2800/52; C12Q 2600/158; A61K 31/454

(57) ABSTRACT

The present disclosure relates to methods and kits for classifying an individual afflicted with multiple myeloma based on the likelihood of response to immunomodulatory drugs (IMiDs), such as thalidomide and lenalidomide. The disclosure further relates to methods of treating an individual afflicted with multiple myeloma with an IMiD and with methods for determining a therapy regime based on the likelihood of response to an IMiD as a result of genetic characteristic of the patient.

### FIELD OF THE INVENTION

The present disclosure relates to methods and kits for classifying an individual afflicted with multiple myeloma based on the likelihood of response to immunomodulatory drugs (IMiDs), such as thalidomide and lenalidomide. The disclosure further relates to methods of treating an individual afflicted with Multiple Myeloma with an IMiD and with methods for determining a therapy regime based on the likelihood of response to an IMiD.

### BACKGROUND OF THE INVENTION

Multiple Myeloma (MM), also known as plasma cell leukemia or Kahler's disease, is a cancer of plasma cells, a type of white blood cell normally responsible for producing antibodies. In MM, collections of abnormal plasma cells accumulate in the bone marrow, where they interfere with the production of normal blood cells. Most cases of MM also feature the production of a paraprotein, an abnormal antibody which can cause kidney problems. Bone lesions and hypercalcemia (high blood calcium levels) are also often encountered.

MM is a heterogeneous disease in terms of genetic background, survival and treatment response, for which several ‘novel agents’ are in development.^{1,2} Despite the fact that the disease remains still incurable at this moment in time, these advances have resulted in a clear improvement in the outcome of MM patients.³ For example, the proteasome inhibitor Bortezomib was shown to provide significantly prolonged Progression Free Survival (PFS), and Overall Survival (OS), when compared against non-Bortezomib containing regimes such as VAD.^{4,5} However, the survival improvements are typically assessed at the group level, disregarding the inhomogeneous nature of the disease. It therefore does not show whether all patients have a small survival benefit, or whether a subgroup of patients has a large benefit. In addition, the high costs and potentially dangerous side effects from these treatments argue for limiting treatment with a drug to only those patients expected to benefit from treatment. The numerous (expensive) drugs on the market and in development for MM, the inhomogeneity of the disease, and the severity of the side effects signify a strong need for predictive markers for MM treatment that would allow personalized treatment to further increase the outcome and quality of life for the individual MM patient

### SUMMARY OF THE INVENTION

In one embodiment, methods are provided for classifying an individual with multiple myeloma based on the likelihood of response to treatment with an immunomodulatory drug (IMiD), the method comprising:

a) determining in a sample from said individual the level of expression of at least one marker selected from Table 11;

b) determining in a sample from said individual the presence of the t(4;14) translocation;

c) determining in a sample from said individual the level of expression of at least one marker in Table 3; and/or

d) determining in a sample from said individual the presence of the t(11;14) translocation;

wherein the individual is classified based on at least one of steps a), b), c), and d).

Preferably, the individual is classified as

i) a likely responder to thalidomide or an analog thereof which is not substituted with NH₂ or CH₃ at the C4 of the phthaloyl ring and a likely responder to a thalidomide analog which is substituted with NH₂ or CH₃ at the C4 of the phthaloyl ring,

ii) a likely responder to a thalidomide analog which is substituted with NH₂ or CH₃ at the C4 of the phthaloyl ring and a likely non-responder to thalidomide or an analog thereof which is not substituted with NH₂ or CH₃ at the C4 of the phthaloyl ring, or

iii) a likely non-responder to a thalidomide analog which is substituted with NH₂ or CH₃ at the C4 of the phthaloyl ring and a likely responder to thalidomide or an analog thereof which is not substituted with NH₂ or CH₃ at the C4 of the phthaloyl ring.

Preferably, the method comprises

a) determining in a sample from said individual the level of expression of at least one markers selected from Table 11 and/or b) determining in a sample from said individual the presence of the t(4;14) translocation; and

c) determining in a sample from said individual the level of expression of at least one marker in Table 3 and/or d) determining in a sample from said individual the presence of the t(11;14) translocation;

wherein the individual is classified based on steps a) and/or b) and on steps c) and/or d).

Preferably, the methods disclosed herein comprise gene expression profiling.

In one embodiment, methods are provided for treating an individual for multiple myeloma comprising

a) determining in a sample from said individual the level of expression of at least one marker selected from Table 11;

b) determining in a sample from said individual the presence of the t(4;14) translocation;

c) determining in a sample from said individual the level of expression of at least one marker in Table 3; and/or

d) determining in a sample from said individual the presence of the t(11;14) translocation;

determining based on steps a), b), c), and/or d) a treatment of the individual, and treating said individual accordingly.

In one embodiment, methods are provided for treating an individual for multiple myeloma comprising administering to an individual in need thereof thalidomide or an analog thereof which is not substituted with NH₂ or CH₃ at the C4 of the phthaloyl ring, wherein said individual is predicted to likely respond to treatment, said prediction being based on the level of expression of at least one marker selected from Table 11, the presence of the t(4;14) translocation, the level of expression of at least one marker selected from Table 3 and/or the presence of the t(11;14) translocation.

In one embodiment, methods are provided for treating an individual for multiple myeloma comprising administering to an individual in need thereof a analog substituted with NH₂ or CH₃ at the C4 of the phthaloyl ring, wherein said individual is predicted to likely respond to treatment, said prediction being based on the level of expression of at least one marker selected from Table 11, the presence of the t(4;14) translocation, the level of expression of at least one marker selected from Table 3 and/or the presence of the t(11;14) translocation.

In one embodiment, thalidomide or an analog thereof which is not substituted with NH₂ or CH₃ at the C4 of the phthaloyl ring is provided for use in the treatment of multiple myeloma in an individual likely to respond to thalidomide treatment, wherein the likelihood of response to thalidomide or the analog thereof is determined by

a) determining in a sample from said individual the level of expression of at least one marker selected from Table 11;

b) determining in a sample from said individual the presence of the t(4;14) translocation;

c) determining in a sample from said individual the level of expression of at least one marker in Table 3; and/or

d) determining in a sample from said individual the presence of the t(11;14) translocation. Preferably, the likelihood of response to thalidomide or the analog thereof is determined by a) determining in a sample from said individual the level of expression of at least one marker selected from Table 11 and/or b) determining in a sample from said individual the presence of the t(4;14) translocation.

Preferably, step a) comprises determining in a sample from said individual the level of expression of at least two markers, wherein at least one marker is selected from Table 11 and at least one marker is selected from Table 11 or Table 12. More preferably, step a) comprises determining the level of expression of the markers from Table 1, the markers from Table 2, and/or the markers from Table 4.

In one embodiment, thalidomide analog which is substituted with NH₂ or CH₃ at the C4 of the phthaloyl ring for use in the treatment of multiple myeloma in an individual likely to respond to the thalidomide analog treatment, and

wherein the likelihood of response to thalidomide analog is determined by

a) determining in a sample from said individual the level of expression of at least one marker selected from Table 11;

b) determining in a sample from said individual the presence of the t(4;14) translocation;

c) determining in a sample from said individual the level of expression of at least one marker in Table 3; and/or

d) determining in a sample from said individual the presence of the t(11;14) translocation. Preferably, the likelihood of response to the thalidomide analog is determined by determining in a sample from said individual the level of expression of at least one marker in Table 3 and/or determining in a sample from said individual the presence of the t(11;14) translocation.

Preferably, the level of marker expression is determined by detection of RNA.

Preferably, the thalidomide analog which is substituted with NH₂ or CH₃ at the C4 of the phthaloyl ring is lenalidomide or pomalidomide.

Preferably, the sample comprises plasma cells.

### BRIEF DESCRIPTION OF THE DRAWINGS

FIG. 1: Kaplan Meier curves showing that the SKY92 is significantly prognostic in the H87 dataset for Progression Free Survival (PFS, left), and Overall Survival (OS, right). Blue: SKY92 High Risk; Red: SKY92 Standard Risk.

FIG. 2: Kaplan Meier curves showing the SKY92 High Risk/Standard Risk split into Treatment arms MPT-T and MPR-R. Data from the H87 cohort and for Overall Survival.

FIG. 3: Kaplan Meier curves showing the Virtual t(4;14), MS Cluster, and iFISH t(4;14) positive and negative groups split into Treatment arms MPT-T and MPR-R. Hazard Ratios were calculated within positive patients between treatment arms, and within negative patients between treatment arms. Data from the H87 cohort and for Overall Survival.

FIG. 4: Kaplan Meier curves showing the Virtual t(11;14), and iFISH t(11;14) positive and negative groups split into Treatment arms MPT-T and MPR-R. Hazard Ratios were calculated within positive patients between treatment arms, and within negative patients between treatment arms. Data from the H87 cohort and for Overall Survival.

FIG. 5: Scatterplots showing the Hazard Ratio (TC4-/TC4sub) in the group identified as positive. Hazard Ratios above 1 indicate a better Overall Survival for the MPR-R treatment when compared against MPT-T. Conversely, a Hazard Ratio of smaller than 1 indicates that the MPT-T treatment has a better Overall Survival when compared against MPR-R. Hazard Ratios larger than 15 were set to 15.

### DETAILED DESCRIPTION OF THE DISCLOSED EMBODIMENTS

Immunomodulatory drugs (IMiDs), such as thalidomide and lenalidomide, may be used in the treatment of MM. It is believed that IMiDs exert their effect, at least in part, by enhancing CD4+ and CD8+ T cell costimulation. Cereblon (CRBN), a Cullin 4 ring E3 ligase complex, has been shown to be a target of IMiDs and low CRBN levels were found to correlate with poor response (or resistance) to IMiDs.

It has been suggested that biomarkers may predict the response of an MM patient to treatment with IMiDs (see, e.g., WO2012125405 and WO2011020839). Surprisingly, the present disclosure demonstrates that it is possible to distinguish the likelihood of response between different IMiDs for particular patient subsets defined by their genetic characteristics. The present disclosure demonstrates that thalidomide and compounds which are structurally related to thalidomide can be categorized in two separate groups of compounds based on the ability to predict responsiveness in these two groups. A first group comprises thalidomide and analogs thereof which are not substituted with NH₂ or CH₃ at the C4 of the phthaloyl ring, herein referred to collectively as “TC4-compounds”. A second group comprises thalidomide analogs which are substituted with NH₂ or CH₃ at the C4 of the phthaloyl ring, herein referred to as “TC4sub compounds”.

In accordance with the methods and kits described herein, a patient may be classified as likely responding (similarly) to a TC4-compound and a TC4sub compound, as likely responding better to a TC4-compound than a TC4sub compound, or as likely responding better to a TC4sub compound than a TC4-compound.

Accordingly, one object of the disclosure is to provide methods and kits that distinguish the response of a patient to a TC4-compound versus the response to a TC4sub compound. Such methods and kits are not only useful for predicting response to an IMiD, but also provide an indication as to which IMiD is likely to be more effective for a particular patient. Accordingly, the methods and kits described herein are also useful in determining a treatment regime.

IMiDs include thalidomide as well as thalidomide analogs. Thalidomide (2-(2,6-dioxopiperidin-3-yl)-1H-isoindole-1,3(2H)-dione) is composed of a glutarimide ring and a phthaloyl ring and has the following chemical structure:

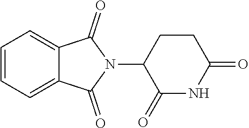 [SMILES: O=C1CCC(N2C(=O)c3ccccc3C2=O)C(=O)N1]

As used herein, a thalidomide analog refers to a compound having the backbone structure of thalidomide (a glutarimide ring and a phthaloyl ring). Such compounds are described, e.g. in US2015/0164877. The thalidomide analogs described herein may include any modification of the thalidomide backbone structure. In preferred embodiments, the thalidomide analog binds to CRBN.

TC4-compounds, as used herein, include thalidomide (which is not substituted at the C4 of the phthaloyl ring) and thalidomide analogs which are not substituted with NH₂ or CH₃ at the C4 of the phthaloyl ring. These analogs include compounds which are not substituted at the C4 of the phthaloyl ring and compounds that contain substitutions such (CH₃)₂, herein referred to collectively as “TC4-compounds”. A preferred TC4-compound of the disclosure is thalidomide.

Preferred TC4sub compounds include lenalidomide and pomalidomide. More preferably the derivative is lenalidomide. Lenalidomide, also known as 3-(4-amino-1-oxo-1,3-dihydro-isoindol-2-yl)-piperidine-2,6-dione (having the tradename Revlimid™) has the following chemical structure:

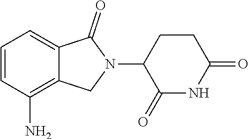 [SMILES: Nc1cccc2c1CN(C1CCC(=O)NC1=O)C2=O]

Pomalidomide, also known as 4-Amino-2-(2,6-dioxopiperidin-3-yl)isoindole-1,3-dione (having the tradenames Imnovid™ and Pomalyst™) has the following chemical structure:

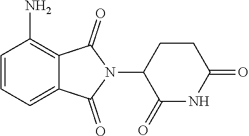 [SMILES: Nc1cccc2c1C(=O)N(C1CCC(=O)NC1=O)C2=O]

In preferred embodiments, the TC4sub compound binds one or more IKAROS transcription factors (e.g., IKZF1 and IKZF3).

While TC4-compounds and TC4sub compounds are both useful in the treatment of MM, these compounds differ in their biological activity, in particular in their ability to promote ubiquitination of the IKAROS family transcription factors by CRBN. As recently described in Fischer et al. (Nature. 2014 Jul. 16 DOI: 10.1038/nature13527), thalidomide, lenalidomide, and pomalidomide all bind similarly to CRBN. However, lenalidomide, pomalidomide, and 2-(2,6-dioxopiperidin-3-yl)-4-methylisoindoline-1,3-dione (a thalidomide analog having a CH₃ substitution at the C4 of the phthaloyl ring) are more efficient at targeting IKAROS transcription factors for degradation by CRBN than thalidomide. While not wishing to be bound by theory, it is likely that the differences in patient response to IMiD treatment described herein are related to the differential targeting of IKAROS transcription factors.

One aspect of the disclosure provides methods for classifying an individual with MM based on the likelihood of response to treatment with an immunomodulatory drug (IMiD). The individual is classified as a likely responder to a TC4-compound and a likely responder to a TC4sub compound, as a likely non-responder to a TC4sub compound and a likely responder to a TC4-compound, or as a likely responder to a TC4sub compound and a likely non-responder to a TC4-compound.

Said methods comprise determining in a sample from said individual:

1. the level of expression of at least one marker selected from Table 1, Table 2, Table 4, Table 11, and Table 12;

2. the presence of the t(4;14) translocation;

3. the level of expression of at least one marker in Table 3;

4. the presence of the t(11;14) translocation; and/or wherein the individual is classified based on at least one of the steps above.

Preferably the method comprises steps 1, 2, 3, and 4. Preferably the method comprises steps 1, 2, and/or 3. Preferably the method comprises steps 1 or 2. Preferably the method comprises steps (1 or 2) and (3 or 4). Preferably the method comprises steps (1 or 2) and 4. Preferably the method comprises steps 3 or 4. Preferably the method comprises step 1. Preferably the method comprises step 2. Preferably the method comprises step 3. Preferably the method comprises step 4. Preferably the method comprises step 5. Preferably the method comprises steps 1 and 3. Preferably the method comprises steps 2 and 3. Preferably the method comprises steps 1 and 4. Preferably the method comprises steps 2 and 4.

As described in the examples, the disclosure demonstrates that the level of expression of at least one marker selected from Table 1, Table 2, Table 4, Table 11, and Table 12 (step 1) can be used to classify whether the individual is a likely responder to a TC4sub compound and a likely non-responder to a TC4-compound or that the individual is a likely responder to a TC4sub compound and a likely responder to a TC4-compound. The Tables list Affymetrix probesets and their corresponding “markers” (genes).

In preferred embodiments, the level of expression of at least two markers selected from Table 1, Table 2, Table 4, Table 11, and Table 12 is determined. In some embodiments, the level of expression of at least three, at least four, at least five, at least six, at least seven, at least eight, at least nine, at least 10, at least 20, or at least 30 markers selected from Table 1-4, Table 11, and Table 12 is determined. In preferred embodiments, the level of expression of at least two markers selected from Table 1 is determined. In preferred embodiments, the level of expression of at least two markers selected from Table 2 is determined. In preferred embodiments, the level of expression of at least two markers selected from Table 4 is determined. In preferred embodiments, the level of expression of at least two markers selected from Table 12 is determined.

In preferred embodiments, the level of expression of all markers from Table 1 is determined. In preferred embodiments, the level of expression of all markers from Table 2 is determined. In preferred embodiments, the level of expression of all markers from Table 4 is determined. In preferred embodiments, the level of expression of all markers from Table 12 is determined.

In more preferred embodiments, the level of expression of at least one marker from Table 11 is determined in the methods. As described herein, Table 11 depicts markers which can each, independently, identify patients that have a higher likelihood of responding to a TC4sub compound than to a TC4-compound.

In some embodiments, the level of expression of at least two markers is determined, wherein at least one marker is selected from Table 11 and at least one marker is selected from Table 11 or Table 12. In some embodiments, the level of expression of at least three markers is determined, wherein at least one marker is selected from Table 11 and at least two markers are selected from Table 11 or Table 12. In some embodiments, the level of expression of at least four markers is determined, wherein at least one marker is selected from Table 11 and at least three markers are selected from Table 11 or Table 12. In some embodiments, the level of expression of at least five markers is determined, wherein at least one marker is selected from Table 11 and at least four markers are selected from Table 11 or Table 12. In some embodiments, the level of expression of at least ten markers is determined, wherein at least one marker is selected from Table 11 and at least nine markers are selected from Table 11 or Table 12.

An individual is classified into one of two groups based on the level of marker expression and whether the level is altered or “differentially expressed” as compared to a reference. Determining the level of expression includes the expression of nucleic acid, preferably mRNA, or the expression of protein. In some embodiments, nucleic acid or protein is purified from the sample and the marker is measured by nucleic acid or protein expression analysis. Preferably, the sample comprises plasma cells. Although a preferred source of plasma cells is a bone marrow sample, other plasma cell containing samples, such as, e.g., blood, may also be used.

Table 1, Table 2, Table 4, Table 11, and Table 12 list Affymetrix DNA probes corresponding to particular genes, i.e., “markers”, as used herein. Marker expression can be measured at the level of nucleic acid or protein.

It is clear to a skilled person, that the term “the level of expression of at least one marker selected from Table 1, Table 2, Table 4, Table 11, and Table 12” refers to the level of nucleic acid corresponding to the probes listed in the table or the corresponding genes they refer to. It is well within the purview of a skilled person to develop additional probes that detect the markers referred to in the tables. The level of nucleic acid expression may be determined by any method known in the art including RT-PCR, quantitative PCR, Northern blotting, gene sequencing, in particular RNA sequencing, and gene expression profiling techniques. Preferably, the level of nucleic acid using a microarray.

Preferably, the nucleic acid is RNA, such as mRNA or pre-mRNA. As is understood by a skilled person, the level of RNA expression determined may be detected directly or it may be determined indirectly, for example, by first generating cDNA and/or by amplifying the RNA/cDNA. The level of expression need not be an absolute value but rather a normalized expression value or a relative value.

It is clear to a skilled person, that in some embodiments the term “the level of expression of at least one marker selected from Table 1, Table 2, Table 4, Table 11, and Table 12” refers to the level of protein corresponding to the probes or the genes they refer to. The level of expression can be determined by any method known in the art including ELISAs, immunocytochemistry, flow cytometry, Western blotting, proteomic, and mass spectrometry.

Preferably, the level of expression refers to a “normalized” level of expression. Normalization is particularly useful when expression is determined based on microarray data. Normalization allows for correction for variation within microarrays and across samples so that data from different chips can be simultaneously analyzed. The robust multi-array analysis (RMA) algorithm may be used to pre-process probe set data into gene expression levels for all samples. (Irizarry R A, et al., Biostatistics (2003) and Irizarry R A, et al., Nucleic Acids Res. (2003)). In addition, Affymetrix's default preprocessing algorithm (MAS 5.0), may also be employed. Additional methods of normalizing expression data are described in US20060136145.

As used herein, the term “differentially-expressed” means that the measured expression level in a subject differs significantly from a reference. The reference may be a single value or a numerical range. It is within the purview of a skilled person to determine the appropriate reference value. In some embodiments, the reference value is a predetermined value. In some embodiments, the reference value is the average of the expression value in a particular patient class. For example, the reference value may be the average of the expression value in the class of patients that are predicted to respond to both a TC4-compound and TC4sub compounds). A reference value may also be in the form of or derived from an equation, see, e.g., equations 1 and 2 herein. In preferred embodiments, the reference may be an m₀ or m₁ value as described herein. The reference may also be several values, e.g., the comparison between an m₀ or m₁ value as described herein. It is within the purview of one skilled in the art to determine whether the expression level in the patient differs “significantly” from a reference.

In an exemplary embodiment, the reference value is determined from the HOVON-87/NMSG-18 study, in which response to thalidomide treatment was compared to lenalidomide treatment in MM patients. It is clear to a skilled person that data from similar studies may also be used.

The strength of the correlation between the expression level of a differentially-expressed gene and a specific patient response class may be determined by a statistical test of significance. For example, a chi square test may be used to assign a chi square value to each differentially-expressed marker, indicating the strength of the correlation of the expression of that marker to a specific patient response class. Similarly, the T-statistics metric and the Wilkins' metric both provide a value or score indicative of the strength of the correlation between the expression of the marker and its specific patient response class. In addition, SAM or PAM analysis tools may be used to determine the strength of correlations.

In some embodiments, the subject expression profile (or rather, the expression level of one or more markers) is compared to the reference expression profile to determine whether the subject expression profile is sufficiently similar to the reference profile. Alternatively, the subject expression profile is compared to a plurality of reference expression profiles to select the reference expression profile that is most similar to the subject expression profile. Any method known in the art for comparing two or more data sets to detect similarity between them may be used to compare the subject expression profile to the reference expression profiles.

In machine learning and statistics, classification refers to identifying to which set of categories a new observation belongs, on the basis of a training set of data containing observations (or instances) whose category membership is known. An algorithm that implements classification, especially in a concrete implementation, is known as a classifier. Many classifiers are known in the art, with linear or non-linear classifier boundaries, such as but not limited to: ClaNC, nearest mean classifier, weighted voting method, simple Bayes classifier, linear discriminant analysis (LDA), quadratic discriminant analysis (QDA), Support Vector Machines (SVM), or the k-nearest neighbor (k-nn) classifier.

In a preferred embodiment, a linear classifier is used in the methods described herein. The ClaNC classifier (Classification to Nearest Centroids) is a preferred linear classifier and is described in detail in the examples. Briefly, for a single MM patient referred to as x, a distance d to each of the two centroids is calculated. Centroids are referred to with 0 and 1 subscripts. The employed distance is the normalized Euclidean distance measure, resulting in a d0 and a d1, formulated as:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} \\ and & Equation 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Equation 2\end{matrix}$$

wherein xᵢ represents the expression level of a particular gene i of the MM patient x, N is the total number of genes or probesets used in the particular classifier, mᵢ is the mean of the centroid for gene or probeset i, and sᵢ the standard deviation of the centroid for gene/probeset i. The MM patient is then assigned to the group with the smallest distance d (i.e. the closest centroid).

Tables 2 and 4 provide exemplary values for m₀, m₁, s₀ and s₁ which may be used as a guideline. It is clear to a skilled person that the values listed in the tables may be rounded off to one or two significant digits. The examples also describe how the expression level of a single marker or a collection of markers classify patient response when using the ClaNC classifier. In preferred embodiments, the ClaNC classifier is used in the methods described herein for markers listed in Tables 2 and Table 4.

The weighted voting method is also a preferred linear classifier and is described in detail in the examples. Briefly, for each marker used, a vote for one or the other class (e.g., responder to a TC4-compound and derivative TC4sub compound or a responder to TC4sub compound and non-responder to a TC4-compound) is determined based on expression level. Each vote is then weighted in accordance with the weighted voting scheme (for example the beta values listed in Table 1), and the weighted votes are summed to determine the winning class for the sample.

In an exemplary embodiment, the following formula may be used to classify an individual:

$$SKY92 ( x ) = {\sum }_{i = 1}^{92} {β}_{i} {x}_{i} \qquad Equation 3$$

where βi represents the weight factor of gene i, and xi represents the expression level of gene i in a patient, x. The beta values are listed in Table 1. However, it is clear to a skilled person that other beta values (i.e. “weights”) may be used. A score above the threshold classifies a patient as a responder to a TC4sub compound and non-responder to a TC4-compound. A score at or below the threshold classifies a patient as a responder to both a TC4sub compound and a TC4-compound.

Table 1 provides exemplary beta values (i.e. “weights”), which may be used as a guideline. However, it is clear to a skilled person that other beta values may be used. In preferred embodiments, the threshold is determined such that the top 15-25%, preferably the top 21.7%, scores of an unselected MM patient cohort fall above the threshold. In the exemplary embodiment disclosed in Example 1, this results in a threshold of 0.7774. However, it is clear to a skilled person that other threshold values may be used. It is also clear to a skilled person that the threshold may be rounded off to one or two significant digits.

In preferred embodiments, the weighted voting method is used in the methods described herein for markers listed in Table 1.

In some embodiments, a subset of the 92 markers of Table 1 is used. In such cases, it is possible to keep the weights of the subset as provided in Table 1 and retrain a new threshold as the top 21.7% of the SKY92 scores. Table 13 provides exemplary threshold values for when only one probeset is used in the methods.

Alternatively, the existing threshold is used and the weight of the discarded markers is redistributed to the remaining genes based on the covariance structure in the training set (HOVON65/GMMG-HD4). Such modifications are within the purview of one of skill in the art. The examples also describe how the expression level of a single marker or a collection of markers classify patient response when using the weighted voting classifier.

In preferred embodiments, the method comprises

a) providing a gene chip comprising probes for the detection of one or more markers selected from Table 1 as described above, in particular including a probe for the detection of a marker that is in both Table 1 and Table 11,

b) contacting the gene chip with nucleic acid obtained directly (e.g. RNA from the sample) or indirectly (e.g., RNA or DNA that has been processed/amplified from the sample) from a sample from a patient,

c) determining the expression levels of the marker(s) in the sample,

d) normalizing the expression levels using mean/variance normalization in order to obtain the normalized expression value

e) multiply the normalized expression value from markers from Table 1 (and those also found in Table 11 or 12) with a beta value (i.e. weight vote, preferably the beta value in Table 1) to obtain the calculated value for an individual probe, f) determine a score by summation of the calculated values of the individual probe(s),

wherein a score above a predetermined threshold (reference value) indicates that the patient is to be classified as a likely responder to derivative TC4sub compound and a likely non-responder to a TC4-compound and a score at or below the predetermined threshold indicates that the patient is to be classified as a likely responder to both a TC4-compound and a TC4sub compound.

In preferred embodiments, the method comprises

a) providing a gene chip comprising probes for the detection of one or more markers selected from Table 2 as described above, in particular including a probe for the detection of a marker that is in both Table 2 and Table 11, as described above,

b) contacting the gene chip with nucleic acid obtained directly (e.g. RNA from the sample) or indirectly (e.g., RNA or DNA that has been processed/amplified from the sample) from a sample from a patient,

c) determining the expression level of the marker(s) in the sample,

d) normalizing the expression level using mean/variance normalization in order to obtain a normalized expression value,

e) solving equations 1 and 2 to obtain d₀ and d₁ values using the normalized expression value from the marker(s) and the m₀, mᵢ, s₀, and sᵢ values from Table 2,

wherein when d₀<d₁, the individual is classified as a likely responder to both a TC4-compound and a TC4sub compound and when d₀ is greater than or equal to d₁, the individual is classified as a likely responder to TC4sub compound and a likely non-responder to a TC4-compound.

In preferred embodiments, the method comprises

a) providing a gene chip comprising probes for the detection of one or more marker selected from Table 4 as described above, in particular including a probe for the detection of a marker that is in both Table 4 and Table 11,

b) contacting the gene chip with nucleic acid obtained directly (e.g. RNA from the sample) or indirectly (e.g., RNA or DNA that has been processed/amplified from the sample) from a sample from a patient,

c) determining the expression level of the marker(s) in the sample,

d) normalizing the expression level using mean/variance normalization in order to obtain a normalized expression value,

e) solving equations 1 and 2 to obtain do and d₁ values using the normalized expression value from the marker(s) and the m₀, mᵢ, s₀, and sᵢ values from Table 4, wherein when d₀<d₁, the individual is classified as a likely responder to both a TC4-compound and a TC4sub compound and when d₀ is greater than or equal to d₁, the individual is classified as a likely responder to TC4sub compound and a likely non-responder to a TC4-compound.

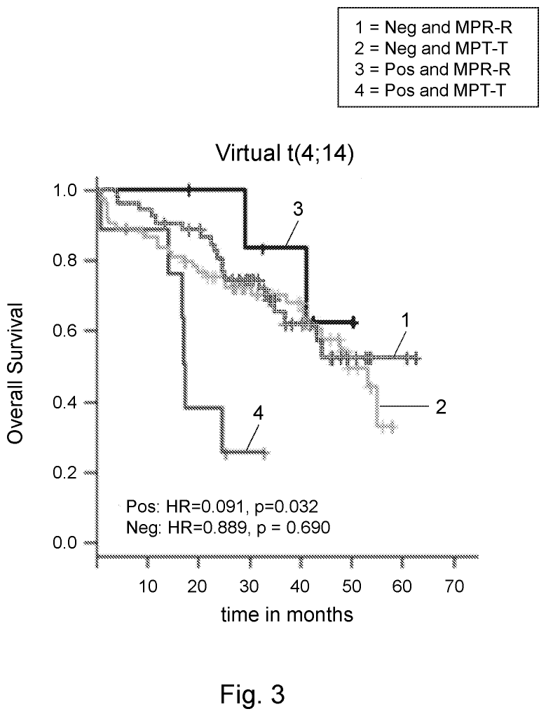

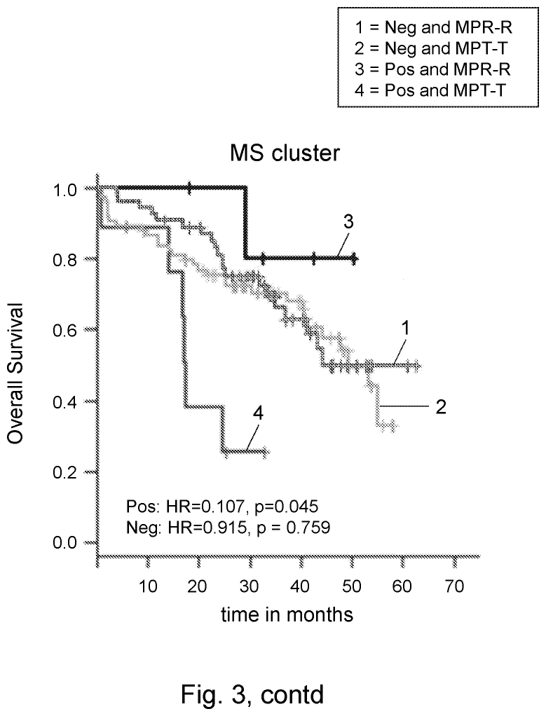

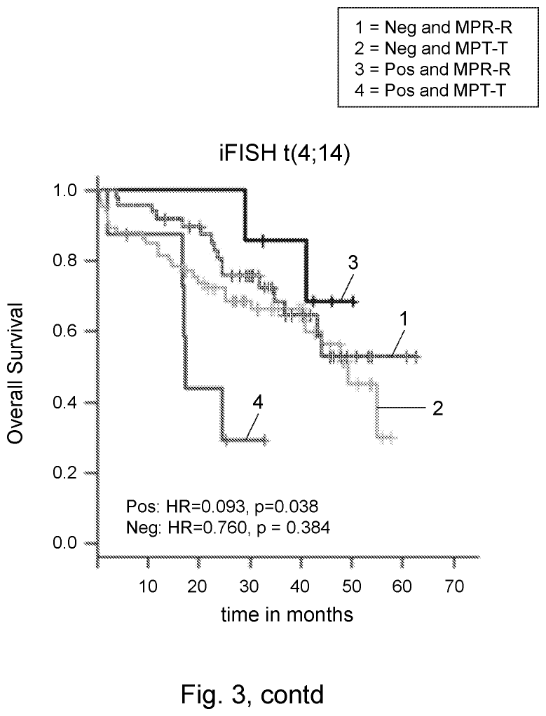

As described in the examples and depicted in FIG. 3, the disclosure demonstrates that the presence of the t(4;14) translocation (step 2) indicates that the individual is a likely responder to a TC4sub compound and a likely non-responder to a TC4-compound. Conversely, the absence of the t(4;14) translocation indicates that the individual is a likely responder to a TC4sub compound and a likely responder to a TC4-compound.

The presence of the t(4;14) translocation can be determined by any means known to a skilled person. As is well known to a skilled person, translocations may be detected by, for example, multiplex ligation dependent probe amplification, by G-banding or R-banding techniques, by comparative genomic hybridization (CGH) such as array-CGH or equivalent DNA copy number aberration (CNA) techniques. In an exemplary embodiment, fluorescence in situ hybridization (FISH) may be used to detect a translocation. Malgeri et al. (Cancer research. 2000; 60 (15): 4058-4061) describes the detection of the t(4;14) translocation using both iFISH and RT-PCR. As it is known that translocation t(4;14) involves FGFR3 and MMSET, the use of markers for FGFR3 and/or MMSET are preferred.

In some embodiments, the presence of the t(4;14) translocation can be determined using a gene expression based profile. Table 2 provides an exemplary list of probe sets which can be used to determine the presence of the t(4;14) translocation.

As described in the examples, the disclosure demonstrates that the level of expression of at least one marker selected from Table 3 (step 3) can be used to classify whether the individual is a likely non-responder to a TC4sub compound and a likely responder to a TC4-compound or that the individual is a likely responder to a TC4sub compound and a likely responder to a TC4-compound.

In preferred embodiments, the level of expression of at least two markers selected from Table 3 or at least three markers selected from Table 3 is determined.

In preferred embodiments, the method comprises

a) providing a gene chip comprising probes for the detection of one or more marker selected from Table 3 as described above,

b) contacting the gene chip with nucleic acid obtained directly (e.g. RNA from the sample) or indirectly (e.g., RNA or DNA that has been processed/amplified from the sample) from a sample from a patient,

c) determining the expression level of the marker(s) in the sample,

d) normalizing the expression level using mean/variance normalization in order to obtain a normalized expression value,

e) solving equations 1 and 2 to obtain do and d₁ values using the normalized expression value from the marker(s) and the m₀, mᵢ, s₀, and sᵢ values from Table 3,

wherein when d₀<d₁, the individual is classified as a likely responder to both a TC4-compound and a TC4sub compound and when d₀ is greater than or equal to d₁, the individual is classified as a likely responder to a TC4-compound and a likely non-responder to a TC4sub compound.

As discussed previously herein, an individual is classified into one of two groups based on the level of marker expression and whether the level is altered or “differentially expressed” as compared to a reference value. In an exemplary embodiment, the reference value is determined from the HOVON-87/NMSG-18 study.

In preferred embodiments, an ClaNC classifier as described herein is used in the methods described herein for the markers listed in Table 3. Table 3 provides exemplary values for m₀, mᵢ, s₀, and sᵢ values which may be used as a guideline. However, it is clear to a skilled person that that values that above or below these numbers will still yield satisfactory results. The examples also describe how the expression level of a single marker or a collection of markers classify patient response when using the ClaNC classifier.

As described in the examples and depicted in FIG. 3, the disclosure demonstrates that the presence of the t(11;14) translocation (step 4) indicates that the individual is a likely non-responder to a TC4sub compound and a likely responder to a TC4-compound. Conversely, the absence of the t(11;14) translocation indicates that the individual is a likely responder to a TC4sub compound and a likely responder to a TC4-compound.

The presence of the t(11;14) translocation can be determined by any means known to a skilled person. As is well known to a skilled person, translocations may be detected by, for example, multiplex ligation dependent probe amplification, by G-banding or R-banding techniques, by comparative genomic hybridization (CGH) such as array-CGH or equivalent DNA copy number aberration (CNA) techniques. In an exemplary embodiment, fluorescence in situ hybridization (FISH) may be used to detect a translocation. As it is known that translocation t(11;14) involves CCND1, the use of markers for CCND1 are preferred (Avet-Loiseau et al. Genes Chromosomes Cancer. 1998 October; 23(2):175-82).

In some embodiments, the presence of the t(11;14) translocation can be determined using a gene expression based profile. Table 3 provides an exemplary list of probe sets which can be used to determine the presence of the t(11;14) translocation.

As used herein, the terms individual, subject, or patient are used interchangeably and include mammals, such as primates and domesticated animals. Preferably said individual is a human.

As used herein, the term “multiple myeloma (MM)” it is meant any type of B-cell malignancy characterized by the accumulation of terminally differentiated B-cells (plasma cells) in the bone marrow, including multiple myeloma cancers which produce light chains of kappa-type and/or light chains of lambda-type; drug resistant multiple myeloma, refractory multiple myeloma or aggressive multiple myeloma, including primary plasma cell leukemia (PCL); and/or optionally including any precursor forms of the disease, including but not limited to benign plasma cell disorders such as MGUS (monoclonal gammopathy of undetermined significance) and/or Waldenstrom's macroglobulinemia (WM, also known as lymphoplasmacytic lymphoma) which may proceed to multiple myeloma; and/or smoldering multiple myeloma (SMM), and/or indolent multiple myeloma, premalignant forms of multiple myeloma which may also proceed to multiple myeloma.

Diagnosis is based on a combination of factors, including the patient's description of symptoms, the doctor's physical examination of the patient, and the results of blood tests and optional x-rays. The diagnosis of multiple myeloma in a subject may occur through any established diagnostic procedure known in the art such as described, e.g., in Rajkumar 2014 (Raikumar Lancet Oncology 2014 Volume 15, Issue 12, e538-e548). Generally, diagnosis of multiple myeloma is made based on either 1) at least 60% of the cells in the bone marrow are plasma cells or 2) the presence of a plasma cell tumor (e.g. identified by biopsy) or least 10% of the cells in the bone marrow are plasma cells; and at least one of the following—high blood calcium level, poor kidney function, low red blood cell counts (anemia), holes in bones from tumor growth found on imaging studies, abnormal area in the bones or bone marrow on an MRI scan, and increase in serum monoclonal Ig.

Smoldering MM refers to early myeloma that is not (yet) causing any (or few) symptoms or problems. Generally, diagnosis of smoldering multiple myeloma is based on one of the following: between 10-60% of the cells in the bone marrow are plasma cells, the presence of high level of monoclonal immunoglobulin (M protein) in the blood, or the presence of high level of light chains in the urine.

In a preferred embodiment, the MM is selected from smoldering MM and symptomatic MM. Preferably, the MM is symptomatic. Symptomatic MM may be defined as, e.g., the presence of a M-protein and/or abnormal free light chain ratio in serum (or urine), and clonal plasma cells in bone marrow or plasmocytoma, and at least 1 myeloma-related dysfunction selected from
calcium >2.65 mmol/l
renal insufficiency (creatinine >177 μmol/l)
anemia (Hb<6.2 mmol/l or >1.25 mmol/l below normal limit) or (Hb<10.0 g/dl or >2.1 g/dl below normal limit)
bone disease (lytic lesions or osteopenia).

The methods and kits disclosed herein are useful for predicting the likelihood for responding to treatment. The term “likelihood” refers to the probability of an event. The term likelihood of response refers to probability that, for example, the rate of tumor progress or tumor cell growth will decrease as a result of treatment. As is clear to a skilled person, the term likelihood of response refers to a probability and not that 100% of all patients that are predicted to respond to a treatment may actually respond.

Response to treatment can be measured by any number of endpoints including t ime-to-disease-progression (TTP), growth size of tumor, and clinical prognostic markers (e.g., level of M protein or percentage of plasma cells in bone marrow). In some embodiments, a responder to treatment demonstrates Complete Response (CR), Stringent Complete Response (sCR), Very Good Partial Response (VGPR), or Partial Response (PR), or Stable Disease (SD), increased Time To Progression (TTP), increased Progression Free Survival (PFS) and Overall Survival (OS); as defined by the International Myeloma Working Group (IMWG). In some embodiments, a responder has a lower hazard rate, e.g. a lower chance of having a certain type of event (disease progression/death) with treatment rather than in the absence of treatment. Preferably, an individual is classified as a likely responder to treatment when the Overall Survival (OS) of the patient is predicted to be longer with treatment rather than in the absence of treatment. OS is defined as the time from a given time-point e.g. the moment of diagnosis or randomization until death from any cause, and is measured in the intent-to-treat population.

Preferably, a “likely responder” and a “likely non-responder” are not defined in absolute terms of response, but rather as a comparison between two IMiD treatments. Preferably, an individual classified as a likely responder to a TC4-compound and a likely responder to a TC4sub compound is predicted to respond similarly to both treatments. Preferably, the predicted Hazard Ratio of TC4-compound/TC4sub compound (the ratio of the two hazard rates) would be around 1 in such cases. Preferably, with a p-value of >0.05.

An individual classified as a likely responder to a TC4-compound and a likely non-responder to a TC4sub compound is predicted to respond better to a TC4-compound treatment. Preferably, the predicted Hazard Ratio of TC4-compound/TC4sub compound (the ratio of the two hazard rates) would be HR<1. Preferably, with a p-value of <0.05. It is clear to a skilled person that other endpoints can be used. For example, for these individuals the TTP or PFS or OS is predicted to be longer when treated with a TC4-compound as compared to a TC4sub compound. In another example, for these individuals the hazard rate is predicted to be lower when treated with a TC4-compound as compared to a TC4sub compound.

Conversely, an individual classified as a likely non-responder to a TC4-compound and a likely responder to a TC4sub compound is predicted to respond better to a TC4sub compound treatment. Preferably, the predicted Hazard Ratio of TC4-compound/TC4sub compound (the ratio of the two hazard rates) would be HR>1. Preferably, with a p-value of <0.05. For example, for these individuals the TTP or PFS or OS is predicted to be shorter when treated with a TC4-compound as compared to a TC4sub compound. In another example, for these individuals the hazard rate is predicted to be higher when treated with a TC4-compound as compared to a TC4sub compound.

As is also clear to a skilled person, the likelihood of response can be a dynamic state. Otherwise stated using a hypothetical example, based on the expression levels of the markers described herein, an individual may be classified at time=t, as a responder to a TC4-compound and a responder to a TC4sub compound. However, at time=t+x, the expression levels of the markers described herein may classify the individual as, for example, a responder to a TC4-compound and a non-responder to a TC4sub compound. As is clear to a skilled person, this change in likelihood of response may be due to effects associated with a change of the genetic profile as a result of the progression of disease or the given treatment. This change may also be due to the development of resistance, for example, if the individual is treated with a TC4sub compound after time=t. Accordingly, the methods disclosed herein for classifying an individual with multiple myeloma based on the likelihood of response to treatment with an IMiD, are also useful for determining or monitoring whether an individual is resistant to or acquired resistance to an IMiD. Accordingly, the individual may be classified right after diagnosis, prior to the start of treatment, during treatment, or after the completion of treatment, e.g. to determine the best maintenance treatment for that individual.

One of the advantages of applying the methods disclosed herein to predict response is that it allows for optimizing a treatment regime. Individuals that are predicted to respond to a particular treatment may be subsequently administered such treatment. Conversely, individuals predicted not to respond to a particular treatment may be administered with an alternative treatment. This can result in a decrease in unnecessary treatments.

Accordingly, the disclosure provides a method for treating an individual for multiple myeloma comprising:

1) determining in a sample from said individual the level of expression of at least one marker selected from Table 1, Table 2, Table 4, Table 11, and Table 12 (preferably the number and combinations of markers as disclosed herein);

2) determining in a sample from said individual the presence of the t(4;14) translocation;

3) determining in a sample from said individual the level of expression of at least one marker in Table 3 (preferably the number and combinations of markers as disclosed herein); and/or

4) determining in a sample from said individual the presence of the t(11;14) translocation;

determining based on steps a), b), c), and/or d) a treatment of the individual, and treating said individual accordingly.

Treatments for MM are well-known to a skilled person and include, e.g., radiation, autologous stem cell transplantation, surgery, and drug therapies. Drug therapies include, among others, thalidomide, thalidomide analogs (e.g., lenalidomide, pomalidomide), proteasome inhibitors (e.g., bortezomib), interferon alfa-2b, and steroids like prednisone, Antibody based therapies, HDAC inhibitors, Alkylating agents, pathway inhibitors etc.

Combination treatments are also well-known to a skilled person and include
doxorubicin/dexamethasone/bortezomib/lenalidomide;
vincristine/doxorubicin/dexamethasone
Rd, lenalidomide/dexamethasone;
MPV, melphalan/prednisone/bortezomib;
VRD, Bortezomib, Lenalidomide, Dexamethasone
KRd, Carfilzomib, Lenalidomide, low dose Dexamethasone
MPT-T, Melphalan, Prednisone, Thalidomide, and Thalidomide maintenance
MPR-R, Melphalan, Prednisone, Lenalidomide, and Lenalidomide maintenance

In some embodiments, the individual is treated with a TC4-compound. Preferably, the individual is treated with induction therapy with melphalan, prednisone and a TC4-compound, followed by a TC4-compound maintenance. In some embodiments, the individual is treated with a TC4sub compound. Preferably, the individual is treated with induction therapy with melphalan, prednisone and a TC4sub compound, followed by TC4sub compound maintenance.

Preferably the treatment method comprises steps 1, 2, and/or 4. Preferably the method comprises steps 1, 2, and/or 3. Preferably the method comprises steps 1 or 2. Preferably the method comprises steps (1 or 2) and (3 or 4). Preferably the method comprises steps (1 or 2) and 4. Preferably the method comprises steps 3 or 4.

These steps provide information regarding the likelihood of patient response. If based on step 1 or 2 the individual is classified as a likely responder to a TC4sub compound and a likely non-responder to a TC4-compound, the individual is preferably not treated with a TC4-compound. Instead the individual may be treated with an alternative MM treatment. In preferred embodiments the MM treatment comprises the use of a TC4sub compound. Accordingly, the disclosure also provides a TC4sub compound for use in the treatment of multiple myeloma, wherein the likelihood of response to the TC4sub compound is determined as disclosed herein.

If based on step 3 or 4 the individual is classified as a likely non-responder to a TC4sub compound and a likely responder to a TC4-compound, the individual is preferably not treated with TC4sub compound. Instead the individual is treated with an alternative MM treatment. In preferred embodiments the MM treatment comprises the use of a TC4-compound. Accordingly, the disclosure also provides a TC4-compound for use in the treatment of multiple myeloma, wherein the likelihood of response to a TC4-compound is determined as disclosed herein.

It is well within the purview of a skilled person to prepare suitable pharmaceutical compositions comprising a TC4-compound or a TC4sub compound. As is clear to a skilled person, treatment of an individual may include administration of such pharmaceutical compositions.

In some embodiments of the disclosure, kits are provided for use in diagnostic, research, and therapeutic applications. Preferably, the disclosure provides kits for classifying an individual with multiple myeloma based on the likelihood of response to treatment with an immunomodulatory drug (IMiD), wherein the kit comprises:

a) means for determining in a sample from said individual the level of expression of at least one marker selected from Table 11;

b) means for determining in a sample from said individual the presence of the t(4;14) translocation;

c) means for determining in a sample from said individual the level of expression of at least one marker in Table 3; and/or

d) means for determining in a sample from said individual the presence of the t(11;14) translocation;

Preferably, the means referred to in step a) or step b) comprise an array of probes, e.g., a microarray. Preferably, the array consists of probes that specifically detect markers selected from Table 1, Table 2, Table 3, Table 4, Table 11 and Table 12. Preferably, at least 5 probes, at least 10 probes, or at least 20 probes are present on the array. In some embodiments, the disclosure provides the use of one or more markers selected from Table 11 as a diagnostic for classifying an individual based on the likelihood of response to treatment with an IMiD, as disclosed herein.

### Definitions

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article.

The invention is further explained in the following examples. These examples do not limit the scope of the invention, but merely serve to clarify the invention.

### EXAMPLES

### Example 1

GEP (gene expression profiling) has enabled the development of signatures, such as the EMC92/SKY92 signature¹⁴, and the GEP clusters (MS, MF, etc.).^{6,15} For prognostic purposes, GEP based markers have been shown to be more robust across cohorts compared to iFISH results.^{16,17} Consequently, they have been integrated into clinical guidelines and consensus papers¹⁸, and currently pave the way for risk stratified treatment approaches in MM. Five GEP markers (SKY92, virtual gain(1q), virtual t(14;16)/t(14;20), cluster CD2, MF cluster) have been previously identified, which distinguish patients with a survival benefit when treated with proteasome inhibitors²¹.

Here we applied GEP on samples from the HOVON-87/NMSG-18 study¹⁹ for comprehensive genetic subtyping. In the HOVON-87/NMSG-18 study, induction therapy with melphalan, prednisone and thalidomide, followed by thalidomide maintenance (MPT-T), was compared with melphalan, prednisone and lenalidomide, followed by lenalidomide maintenance (MPR-R). The data shows that patients that are identified to belong to the genetic subtype SKY92, virtual t(4;14), MS cluster, or iFISH t(4;14), have a survival benefit from Lenalidomide induction and maintenance treatment compared to thalidomide induction and maintenance treatment and therefore should be preferentially treated with a Lenalidomide regime. In other words, SKY92 positive patients should be treated with MPR-R rather than MPT-T. Conversely, virtual t(11;14) patients have a survival benefit from thalidomide induction and maintenance treatment compared to lenalidomide induction and maintenance treatment and therefore should be preferentially treated with a thalidomide regime. In other words, virtual t(11;14) positive cases should be treated with MPT-T rather than MPR-R.

Materials and Methods

The HOVON-87/NMSG-18 trial (EudraCTnr.: 2007-004007-34) is a phase 3 trial for elderly MM patients (age 65 and older, or age <65 and transplant in-eligible) in which induction therapy with melphalan, prednisone and thalidomide, followed by thalidomide maintenance, was compared with melphalan, prednisone and lenalidomide, followed by lenalidomide maintenance (MPT-T vs. MPR-R).

Interphase FISH on isolated CD138-positive plasma cells was performed according to the EMN guidelines (Ross et al., Haematolologica 2012 97:1272), in order to determine the presence of t(4;14) and t911:14).

Gene Expression Profiles (GEP) were assessed from n=143 samples enrolled in this HOVON-87/NMSG-18 trial using the MMprofiler. Out of these 143 patients, 83 were from the MPT-T arm, and 60 from the MPR-R arm. The GEP data were normalized as described in Van Vliet et al. 2014²⁰. Subsequently, SKY92 (=EMC92) scores were calculated as described in Kuiper et al 2012¹⁴. Briefly, the SKY92 is a summation of the weighted expression of 92 probe sets (see Table 1). This signature constitutes a linear model, expressed in the following formula:

$$SKY92 ( x ) = {\sum }_{i = 1}^{92} {β}_{i} {x}_{i} \qquad Equation 3$$

where βi represents the weight factor of gene i, and xᵢ represents the expression level of gene i in a patient. Based on their SKY92 score, patients were split into two groups, those above the threshold of 0.7774 were classified as positive (High Risk), and those below the threshold as negative (Standard Risk)¹⁴. When using subsets of the 92 probesets, it is possible to keep the weights of that subset as provided in Table 1, and retrain a new threshold as the top 21.7% of the SKY92 scores, or to redistribute the weight of discarded genes to the remaining genes based on the covariance structure in the training set (HOVON-65/GMMG-HD4), and still use the existing threshold of 0.7774.

For the virtual t(4;14), virtual t(11;14), and MS cluster markers, classifiers have been trained that employ a selection of probe sets (see Table 2, 3, and 4) that enable the distinction of whether a subject does have that characteristic (positive or 1) or does not have that characteristic (negative or 0). Specifically, the Classification to Nearest Centroids method was used (ClaNC)²², known in the art as linear classifiers (nearest mean classifier, LDA, or similar). The method uses the mean and standard deviation of each class to classify a new patient. For a new patient, the normalized Euclidean distances are calculated to each of the two classes, as defined by:

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} & Equation 1 \\ {d}_{1} ( x ) = \sqrt{{\sum }_{i = 1}^{N} \frac{{( {x}_{i} - {m}_{1 , i} )}^{2}}{{s}_{1 , i}^{2}}} & Equation 2\end{matrix}$$

where xᵢ represents the expression level of gene i in a patient, N represents the total number of probe sets, m_{1,i} represents the mean of centroid 1 for gene i and s_{1,i} represents the standard deviation of centroid 1 for gene i. These values can be found per probe set for each marker in. Using d₁ and d₀, a patient is assigned to the nearest class. For example, when d₁=3 and d₀=1, the patient will be assigned to class 0 because distance d₁ is greater than distance d₀. See Example 1a below for a detailed description. When using subsets of the probesets provided in the Tables, the procedure remains the same, i.e. when only two probesets are used the formulas in Equation 1 and 2 are only applied using those two probesets. The evaluation of d0 and d1 remains the same.

Survival curves were plotted using the Kaplan-Meier method. The Cox proportional hazards model was used to assess Hazard Ratios (HR) between groups of patients.

TABLE 1
SKY92 probe sets and weights

| Probesets | Beta | Gene Symbol |
|---|---|---|
| 200701_at | −0.0210 | NPC2 |
| 200775_s_at | 0.0163 | HNRNPK /// MIR7-1 |
| 200875_s_at | 0.0437 | MIR1292 /// NOP56 /// |
| | | SNORD110 /// SNORD57 /// |
| | | SNORD86 |
| 200933_x_at | −0.0323 | RPS4X |
| 201102_s_at | 0.0349 | PFKL |
| 201292_at | −0.0372 | TOP2A |
| 201307_at | 0.0165 | SEPT11 |
| 201398_s_at | −0.0254 | TRAM1 |
| 201555_at | −0.0052 | MCM3 |
| 201795_at | 0.0067 | LBR |
| 201930_at | −0.0090 | MCM6 |
| 202107_s_at | 0.0225 | MCM2 |
| 202322_s_at | 0.0129 | GGPS1 |
| 202532_s_at | −0.0006 | DHFR |
| 202542_s_at | 0.0870 | AIMP1 |
| 202553_s_at | 0.0054 | SYF2 |
| 202728_s_at | −0.1105 | LTBP1 |
| 202813_at | 0.0548 | TARBP1 |
| 202842_s_at | −0.0626 | DNAJB9 |
| 202884_s_at | 0.0714 | PPP2R1B |
| 203145_at | −0.0002 | SPAG5 |
| 204026_s_at | 0.0046 | ZWINT |
| 204379_s_at | 0.0594 | FGFR3 |
| 205046_at | 0.0087 | CENPE |
| 206204_at | 0.0477 | GRB14 |
| 207618_s_at | 0.0746 | BCS1L |
| 208232_x_at | −0.0493 | NRG1 |
| 208667_s_at | −0.0390 | ST13 |
| 208732_at | −0.0618 | RAB2A |
| 208747_s_at | −0.0874 | C1S |
| 208904_s_at | −0.0334 | RPS28 |
| 208942_s_at | −0.0997 | SEC62 |
| 208967_s_at | 0.0113 | AK2 |
| 209026_x_at | 0.0255 | TUBB |
| 209683_at | −0.0561 | FAM49A |
| 210334_x_at | 0.0175 | BIRC5 |
| 211714_x_at | 0.0221 | TUBB |
| 211963_s_at | 0.0303 | ARPC5 |
| 212055_at | 0.0384 | TPGS2 |
| 212282_at | 0.0530 | TMEM97 |
| 212788_x_at | −0.0164 | FTL |
| 213002_at | −0.0418 | MARCKS |
| 213007_at | −0.0106 | FANCI |
| 213350_at | 0.0056 | RPS11 |
| 214150_x_at | −0.0349 | ATP6V0E1 |
| 214482_at | 0.0861 | ZBTB25 |
| 214612_x_at | 0.0496 | MAGEA6 |
| 215177_s_at | −0.0768 | ITGA6 |
| 215181_at | −0.0342 | CDH22 |
| 216473_x_at | −0.0576 | DUX2 /// DUX4 /// DUX4L2 /// |
| | | DUX4L3 /// DUX4L4 /// DUX4L5 /// |
| | | DUX4L6 /// DUX4L7 /// |
| | | LOC100288627 /// LOC100288657 /// |
| | | LOC652119 |
| 217548_at | −0.0423 | LOC100129502 |
| 217728_at | 0.0773 | S100A6 |
| 217732_s_at | −0.0252 | ITM2B |
| 217824_at | −0.0041 | UBE2J1 |
| 217852_s_at | 0.0008 | ARL8B |
| 218355_at | 0.0116 | KIF4A |
| 218365_s_at | 0.0035 | DARS2 |
| 218662_s_at | −0.0176 | NCAPG |
| 219510_at | −0.0097 | POLQ |
| 219550_at | 0.0559 | ROBO3 |
| 220351_at | 0.0420 | CCRL1 |
| 221041_s_at | −0.0520 | SLC17A5 |
| 221606_s_at | 0.0208 | HMGN5 |
| 221677_s_at | 0.0126 | DONSON |
| 221755_at | 0.0396 | EHBP1L1 |
| 221826_at | 0.0200 | ANGEL2 |
| 222154_s_at | 0.0154 | SPATS2L |
| 222680_s_at | 0.0205 | DTL |
| 222713_s_at | 0.0278 | FANCF |
| 223381_at | −0.0070 | NUF2 |
| 223811_s_at | 0.0556 | GET4 /// SUN1 |
| 224009_x_at | −0.0520 | DHRS9 |
| 225366_at | 0.0140 | PGM2 |
| 225601_at | 0.0750 | HMGB3 |
| 226217_at | −0.0319 | SLC30A7 |
| 226218_at | −0.0644 | IL7R |
| 226742_at | −0.0345 | SAR1B |
| 228416_at | −0.0778 | ACVR2A |
| 230034_x_at | −0.0330 | MRPL41 |
| 231210_at | 0.0093 | C11orf85 |
| 231738_at | 0.0686 | PCDHB7 |
| 231989_s_at | 0.0730 | 61E3.4 /// LOC100132247 /// |
| | | LOC100271836 /// LOC100652992 /// |
| | | LOC613037 /// LOC728888 /// |
| | | NPIPL3 /// SLC7A5P1 /// SMG1P1 |
| 233399_x_at | −0.0184 | TMED10P1 /// ZNF252 |
| 233437_at | 0.0446 | GABRA4 |
| 238116_at | 0.0661 | DYNLRB2 |
| 238662_at | 0.0490 | ATPBD4 |
| 238780_s_at | −0.0529 | — |
| 239054_at | −0.1088 | SFMBT1 |
| 242180_at | −0.0585 | TSPAN16 |
| 243018_at | 0.0407 | — |
| 38158_at | 0.0423 | ESPL1 |
| AFFX-HUMISGF3A/ | 0.0525 | STAT1 /// STAT1 |
| M97935_MA_at | | |

Positive beta values (i.e., weight values) indicate that increased expression of said gene over a reference value indicates a positive contribution towards the SKY92 score, as a consequence a larger chance of being above the threshold, or rather that the patient likely responds to MPR-R and does not likely respond to MPT-T. Conversely, positive beta values indicate that decreased expression of said gene over a reference value indicates a negative, contribution towards the SKY92 score, as a consequence a larger chance of being below the threshold, or rather that the patient likely responds to MPR-R and to MPT-T.

Negative beta values indicate that decreased expression of said gene over a reference value indicates a positive contribution towards the SKY92 score, as a consequence a larger chance of being above the threshold, or rather that the patient likely responds to MPR-R and does not likely respond to MPT-T. Conversely, negative beta values indicate that increased expression of said gene over a reference value indicates a negative, contribution towards the SKY92 score, as a consequence a larger chance of being below the threshold or rather that the patient likely responds to MPR-R and to MPT-T.

TABLE 2
Virtual t(4;14) probe sets

| | t(4:14) negative | | t(4:14) positive | | Gene |
|---|---|---|---|---|---|
| Probeset | m0 | s0 | m1 | s1 | symbol |
| 204379_s_at | −0.25462 | 0.530071 | 1.537652 | 1.496546 | FGFR3 |
| 205131_x_at | −0.27838 | 0.842607 | 1.175988 | 0.843791 | CLEC11A |
| 205830_at | −0.19975 | 0.870945 | 1.092877 | 0.621865 | CLGN |
| 211709_s_at | −0.22553 | 0.727177 | 1.384303 | 1.030579 | CLEC11A |
| 212148_at | −0.39946 | 0.769413 | 1.220048 | 0.760111 | PBX1 |
| 212151_at | −0.36575 | 0.844361 | 1.168239 | 0.81162 | PBX1 |
| 212813_at | −0.26267 | 0.766274 | 1.243431 | 0.814053 | JAM3 |
| 217867_x_at | −0.20611 | 0.822701 | 1.49116 | 0.815068 | BACE2 |
| 221261_x_at | −0.14034 | 0.952932 | 1.158952 | 0.395102 | MAGED4 /// |
| | | | | | MAGED4B /// |
| | | | | | SNORA11D /// |
| | | | | | SNORA11E |
| 222258_s_at | −0.26969 | 0.87769 | 1.168436 | 0.604416 | SH3BP4 |
| 222777_s_at | −0.3524 | 0.622727 | 1.631826 | 0.949852 | WHSC1 |
| 222778_s_at | −0.42306 | 0.627198 | 1.518182 | 0.980568 | WHSC1 |
| 223313_s_at | −0.10729 | 0.940715 | 1.210379 | 0.540292 | MAGED4 /// |
| | | | | | MAGED4B /// |
| | | | | | SNORA11D /// |
| | | | | | SNORA11E |
| 223472_at | −0.34184 | 0.740682 | 1.200404 | 0.998955 | WHSCI |
| 223822_at | −0.20027 | 0.797071 | 1.544708 | 0.887078 | SUSD4 |
| 227084_at | −0.26801 | 0.876058 | 1.259394 | 0.786791 | DTNA |
| 227290_at | −0.22604 | 0.8319 | 1.259377 | 0.703202 | LOC100509498 |
| 227434_at | −0.17149 | 0.806988 | 1.410106 | 0.837651 | WBSCR17 |
| 227692_at | −0.2828 | 0.818394 | 1.232278 | 0.846703 | GNAI1 |

TABLE 3
Virtual t(11; 14) probe sets

| | t(11:14) negative | | t(11:14) positive | | Gene |
|---|---|---|---|---|---|
| Probeset | m0 | s0 | m1 | s1 | symbol |
| 208711_s_at | −0.32007 | 0.740631 | 1.443502 | 0.451984 | CCND1 |
| 208712_at | −0.19407 | 0.841579 | 1.155986 | 0.262512 | CCND1 |
| 235518_at | −0.24236 | 0.920001 | 1.162315 | 0.70785 | SLC8A1 |

TABLE 4
MS cluster probe sets

| | Non-MS | | MS | | Gene |
|---|---|---|---|---|---|
| Probeset | m0 | s0 | m1 | s1 | symbol |
| 1553105_s_at | −0.17941 | 0.863533 | 1.574718 | 0.791573 | DSG2 |
| 1557780_at | −0.18413 | 0.839616 | 1.606693 | 0.826452 | — |
| 204066_s_at | −0.15171 | 0.92486 | 1.358666 | 0.453127 | AGAP1 |
| 204379_s_at | −0.23285 | 0.6569 | 1.898836 | 1.376235 | FGFR3 |
| 205559_s_at | −0.16509 | 0.890517 | 1.471314 | 0.572184 | PCSK5 |
| 211709_s_at | −0.18311 | 0.879425 | 1.524201 | 0.638017 | CLEC11A |
| 212190_at | −0.16699 | 0.896555 | 1.437085 | 0.646261 | SERPINE2 |
| 212686_at | −0.16492 | 0.926994 | 1.357986 | 0.410138 | PPM1H |
| 212771_at | −0.1518 | 0.940981 | 1.381678 | 0.306368 | FAM171A1 |
| 214156_at | −0.19327 | 0.893119 | 1.489983 | 0.543743 | MYRIP |
| 217867_x_at | −0.17452 | 0.8793 | 1.609823 | 0.392832 | BACE2 |
| 217901_at | −0.18751 | 0.880607 | 1.543465 | 0.614766 | DSG2 |
| 222258_s_at | −0.16574 | 0.922573 | 1.384121 | 0.516469 | SH3BP4 |
| 222777_s_at | −0.23881 | 0.712218 | 2.147318 | 0.565077 | WHSC1 |
| 222778_s_at | −0.2283 | 0.705499 | 2.120403 | 0.661262 | WHSC1 |
| 223472_at | −0.18119 | 0.846357 | 1.654937 | 0.632111 | WHSC1 |
| 223822_at | −0.18887 | 0.832638 | 1.590486 | 0.823419 | SUSD4 |
| 227084_at | −0.18523 | 0.880019 | 1.536949 | 0.498332 | DTNA |
| 227692_at | −0.16209 | 0.891145 | 1.469242 | 0.658789 | GNAI1 |
| 238116_at | −0.18331 | 0.845629 | 1.654202 | 0.694305 | DYNLRB2 |

### Example 1a: Method for Determining Whether a Subject Belongs to the MS Cluster Using the ClaNC Method

Fictitious data (fable 5) is used as an example for the classification method, using 2 genes for simplicity, to predict whether a sample belongs to MS or non-MS type. In the column “Example patient data”, the measured expression levels are shown for both genes.

TABLE 5
m and s values for the first two probe sets of the MS cluster
and the example patient data used in example 1. All values are
rounded to 3 decimals for the purpose of the example. The last
two columns are the results of the classification process.

| | | | | | Example | | |
|---|---|---|---|---|---|---|---|
| | Non-MS | | MS | | patient | | |
| Probe set | m0 | s0 | m1 | s1 | data | d₀ | d₁ |
| Probe set 1 | −0.127 | 0.868 | 1.936 | 0.939 | 0.121 | 1.015 | 2.359 |
| Probe set 2 | −0.084 | 0.936 | 1.707 | 0.650 | 0.828 | | |

The d₀ and the d₁ were calculated using the values in Table, and Equations 1 and 2. The worked out formulas are shown in Equation and Equation.

$$\begin{matrix}{d}_{0} ( x ) = \sqrt{{\sum }_{i = 1}^{N} \frac{{( {x}_{i} - {m}_{0 , i} )}^{2}}{{s}_{0 , i}^{2}}} = \sqrt{\frac{{( {x}_{1} - {m}_{0 , 1} )}^{2}}{{s}_{0 , 1}^{2}} + \frac{{( {x}_{2} - {m}_{0 , 2} )}^{2}}{{s}_{0 , 2}^{2}}} = \sqrt{\frac{{( .121 -- 0.127 )}^{2}}{{0.868}^{2}} + \frac{{( 0.828 -- 0.084 )}^{2}}{{0.936}^{2}}} = \sqrt{0.082 + 0.949} = 1.015 & Equation 4 \\ {d}_{1} ( x ) = \sqrt{{\sum }_{i = 1}^{N} \frac{{( {x}_{i} - {m}_{1 , i} )}^{2}}{{s}_{1 , i}^{2}}} = \sqrt{\frac{{( {x}_{1} - {m}_{1 , 1} )}^{2}}{{s}_{1 , 1}^{2}} + \frac{{( {x}_{2} - {m}_{1 , 2} )}^{2}}{{s}_{1 , 2}^{2}}} = \sqrt{\frac{{( 0.121 - 1.936 )}^{2}}{{0.939}^{2}} + \frac{{( 0.828 - 1.707 )}^{2}}{{0.650}^{2}}} = \sqrt{3.736 + 1.829} = 2.359 & Equation 5\end{matrix}$$

The next step is to compare the d₀ and d₁ values. When d₀<d₁ is true, the new patient will be assigned to class 0. If d₀>d₁ is true, the new patient will be assigned to class 1. Here d₀<d₁ is true, which means the new patient is placed in the 0 class (non-MS).

### Example 1b: Method for Determining Whether a Subject Belongs to the SKY92 Positive or SKY92 Negative Group

Fictitious data (fable 6) is used as an example for the SKY92 classification method, to determine whether a sample belongs to SKY92 positive or SKY92 negative. In the column “Example patient data”, the measured expression levels xᵢ are shown for all 92 genes. For each gene the xᵢ is multiplied by the βi, for which the result is provided in a column in Table 6. Subsequently those values are summed up, providing the SKY92(x)=−0.4455. This value is then compared to the threshold of 0.7774, and since it is lower than the threshold, the patient is determined to be SKY92 negative.

TABLE 6
Fictitious data (x) from an example patient for all 92 genes from
the SKY92 signature, the betas of all genes, the result obtained after
multiplication of betas and xᵢ values, and at the bottom of the
Table the summation of all those values (SKY92(x)).

| | | x (Example | |
|---|---|---|---|
| | | patient | Beta * |
| Probesets | Beta | data) | x |
| 200701_at | −0.0210 | 0.1049 | −0.0022 |
| 200775_s_at | 0.0163 | 0.7223 | 0.0118 |
| 200875_s_at | 0.0437 | 2.5855 | 0.1130 |
| 200933_x_at | −0.0323 | −0.6669 | 0.0215 |
| 201102_s_at | 0.0349 | 0.1873 | 0.0065 |
| 201292_at | −0.0372 | −0.0825 | 0.0031 |
| 201307_at | 0.0165 | −1.9330 | −0.0319 |
| 201398_s_at | −0.0254 | −0.4390 | 0.0111 |
| 201555_at | −0.0052 | −1.7947 | 0.0093 |
| 201795_at | 0.0067 | 0.8404 | 0.0056 |
| 201930_at | −0.0090 | −0.8880 | 0.0080 |
| 202107_s_at | 0.0225 | 0.1001 | 0.0023 |
| 202322_s_at | 0.0129 | −0.5445 | −0.0070 |
| 202532_s_at | −0.0006 | 0.3035 | −0.0002 |
| 202542_s_at | 0.0870 | −0.6003 | −0.0522 |
| 202553_s_at | 0.0054 | 0.4900 | 0.0026 |
| 202728_s_at | −0.1105 | 0.7394 | −0.0817 |
| 202813_at | 0.0548 | 1.7119 | 0.0938 |
| 202842_s_at | −0.0626 | −0.1941 | 0.0122 |
| 202884_s_at | 0.0714 | −2.1384 | −0.1527 |
| 203145_at | −0.0002 | −0.8396 | 0.0002 |
| 204026_s_at | 0.0046 | 1.3546 | 0.0062 |
| 204379_s_at | 0.0594 | −1.0722 | −0.0637 |
| 205046_at | 0.0087 | 0.9610 | 0.0084 |
| 206204_at | 0.0477 | 0.1240 | 0.0059 |
| 207618_s_at | 0.0746 | 1.4367 | 0.1072 |
| 208232_x_at | −0.0493 | −1.9609 | 0.0967 |
| 208667_s_at | −0.0390 | −0.1977 | 0.0077 |
| 208732_at | −0.0618 | −1.2078 | 0.0746 |
| 208747_s_at | −0.0874 | 2.9080 | −0.2542 |
| 208904_s_at | −0.0334 | 0.8252 | −0.0276 |
| 208942_s_at | −0.0997 | 1.3790 | −0.1375 |
| 208967_s_at | 0.0113 | −1.0582 | −0.0120 |
| 209026_x_at | 0.0255 | −0.4686 | −0.0119 |
| 209683_at | −0.0561 | −0.2725 | 0.0153 |
| 210334_x_at | 0.0175 | 1.0984 | 0.0192 |
| 211714_x_at | 0.0221 | −0.2779 | −0.0061 |
| 211963_s_at | 0.0303 | 0.7015 | 0.0213 |
| 212055_at | 0.0384 | −2.0518 | −0.0788 |
| 212282_at | 0.0530 | −0.3538 | −0.0188 |
| 212788_x_at | −0.0164 | −0.8236 | 0.0135 |
| 213002_at | −0.0418 | −1.5771 | 0.0659 |
| 213007_at | −0.0106 | 0.5080 | −0.0054 |
| 213350_at | 0.0056 | 0.2820 | 0.0016 |
| 214150_x_at | −0.0349 | 0.0335 | −0.0012 |
| 214482_at | 0.0861 | −1.3337 | −0.1148 |
| 214612_x_at | 0.0496 | 1.1275 | 0.0559 |
| 215177_s_at | −0.0768 | 0.3502 | −0.0269 |
| 215181_at | −0.0342 | −0.2991 | 0.0102 |
| 216473_x_at | −0.0576 | 0.0229 | −0.0013 |
| 217548_at | −0.0423 | −0.2620 | 0.0111 |
| 217728_at | 0.0773 | −1.7502 | −0.1353 |
| 217732_s_at | −0.0252 | −0.2857 | 0.0072 |
| 217824_at | −0.0041 | −0.8314 | 0.0034 |
| 217852_s_at | 0.0008 | −0.9792 | −0.0008 |
| 218355_at | 0.0116 | −1.1564 | −0.0134 |
| 218365_s_at | 0.0035 | −0.5336 | −0.0019 |
| 218662_s_at | −0.0176 | −2.0026 | 0.0352 |
| 219510_at | −0.0097 | 0.9642 | −0.0094 |
| 219550_at | 0.0559 | 0.5201 | 0.0291 |
| 220351_at | 0.0420 | −0.0200 | −0.0008 |
| 221041_s_at | −0.0520 | −0.0348 | 0.0018 |
| 221606_s_at | 0.0208 | −0.7982 | −0.0166 |
| 221677_s_at | 0.0126 | 1.0187 | 0.0128 |
| 221755_at | 0.0396 | −0.1332 | −0.0053 |
| 221826_at | 0.0200 | −0.7145 | −0.0143 |
| 222154_s_at | 0.0154 | 1.3514 | 0.0208 |
| 222680_s_at | 0.0205 | −0.2248 | −0.0046 |
| 222713_s_at | 0.0278 | −0.5890 | −0.0164 |
| 223381_at | −0.0070 | −0.2938 | 0.0021 |
| 223811_s_at | 0.0556 | −0.8479 | −0.0471 |
| 224009_x_at | −0.0520 | −1.1201 | 0.0582 |
| 225366_at | 0.0140 | 2.5260 | 0.0354 |
| 225601_at | 0.0750 | 1.6555 | 0.1242 |
| 226217_at | −0.0319 | 0.3075 | −0.0098 |
| 226218_at | −0.0644 | −1.2571 | 0.0810 |
| 226742_at | −0.0345 | −0.8655 | 0.0299 |
| 228416_at | −0.0778 | −0.1765 | 0.0137 |
| 230034_x_at | −0.0330 | 0.7914 | −0.0261 |
| 231210_at | 0.0093 | −1.3320 | −0.0124 |
| 231738_at | 0.0686 | −2.3299 | −0.1598 |
| 231989_s_at | 0.0730 | −1.4491 | −0.1058 |
| 233399_x_at | −0.0184 | 0.3335 | −0.0061 |
| 233437_at | 0.0446 | 0.3914 | 0.0175 |
| 238116_at | 0.0661 | 0.4517 | 0.0299 |
| 238662_at | 0.0490 | −0.1303 | −0.0064 |
| 238780_s_at | −0.0529 | 0.1837 | −0.0097 |
| 239054_at | −0.1088 | −0.4762 | 0.0518 |
| 242180_at | −0.0585 | 0.8620 | −0.0504 |
| 243018_at | 0.0407 | −1.3617 | −0.0554 |
| 38158_at | 0.0423 | 0.4550 | 0.0192 |
| AFFX- | 0.0525 | −0.8487 | −0.0446 |
| HUMISGF3A/M97935_MA_at | | | |

| | |
|---|---|
| SKY92(x) = Sum(Beta * x) = | −0.4455 |

Results

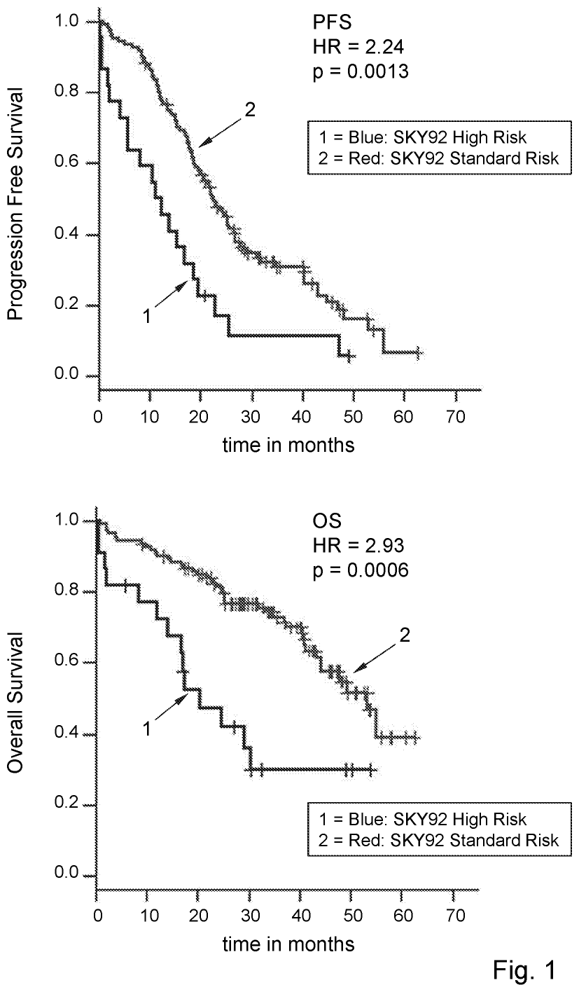

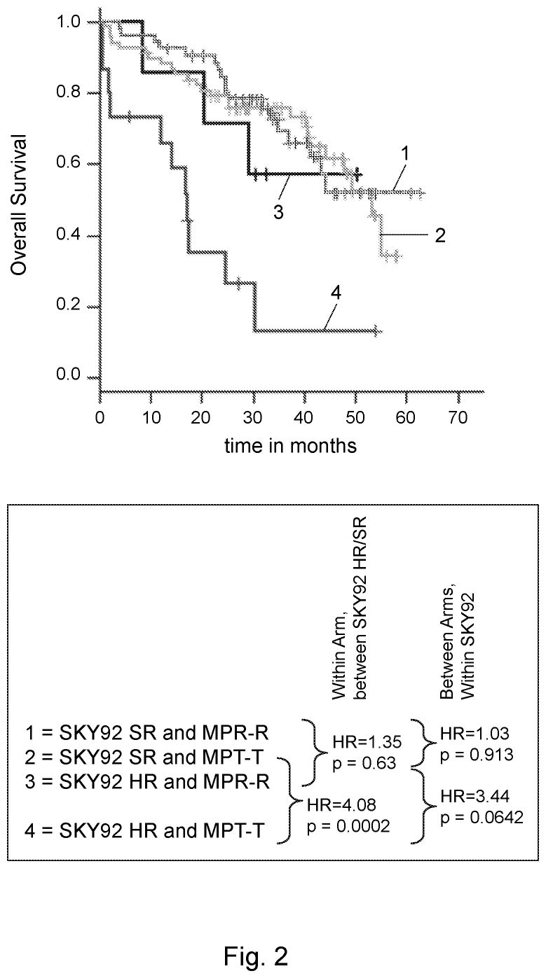

Using the SKY92 signature 22/143 patients were identified as high risk (15.4%). The median overall survival (OS) for high risk patients was 21 months, compared to 53 months for standard risk patients (hazard ratio (HR): 2.9 (95% confidence interval (CI): 1.6-5.4; p=5.6×10-4)). The median progression free survival (PFS) in the high risk and standard risk groups were 12 months and 23 months, respectively (HR: 2.2 (95% CI: 1.4-3.7; p=1.2×10-3)). See FIG. 1. Combining the 2 SKY92 groups and 2 treatment arms results in 4 groups of patients. As can be seen in FIG. 2, for OS there is a significantly different Hazard Ratio between SKY92 SR and SKY92 HR in the MPT-T arm (HR=4.1, p=0.0002), but not in the MPR-R arm (HR=1.35, p=0.63). Comparing the two treatment arms in the SKY92 High Risk group shows that those patients have longer Overall Survival when given MPR-R (HR=3.4, p=0.06). Conversely, in the SKY92 SR group there is no difference between the treatment arm (HR=1.0, p=0.93). These observations support the use of the SKY92 marker to identify a subgroup that benefits from a specific treatment over another treatment whereas the negative cases do not have that treatment benefit. Therefore, the marker can be used to predict specific therapy effectiveness in a subgroup of patients i.e. as a means to determine an MM patient's preferential treatment.

The “Virtual t(4;14)” marker is highly congruent with iFISH t(4;14), and is associated with the MS cluster. These markers are not prognostic in this clinical study, as there is no survival difference between the positive and negative patient groups for this marker (respectively: HR=1.68, p=0.18; HR=1.34, p=0.47; HR=1.63, p=0.23). However, when splitting the positive patients by treatment arm, there is a significant OS advantage when they are treated with MPR-R as opposed to MPT-T (respectively:

HR=0.091, p=0.032; HR=0.093, p=0.038; HR=0.107, p=0.045), whereas there is no difference in the marker negative group (respectively: HR=0.889, p=0.690; HR=0.760, p=0.384; HR=0.915, p=0.759). See FIG. 3. These observations support the use of the Virtual t(4;14), iFISH t(4;14), and the MS cluster as predictive marker, i.e. as a means to determine an MM patient's preferential treatment. Positive cases for either of these three markers iFISH t(4;14), virtual t(4;14) or the MS cluster have a benefit from MPR-R treatment over MPT-T treatment whereas the patients negative for these markers do not have a survival difference when treated with either of the two treatments.

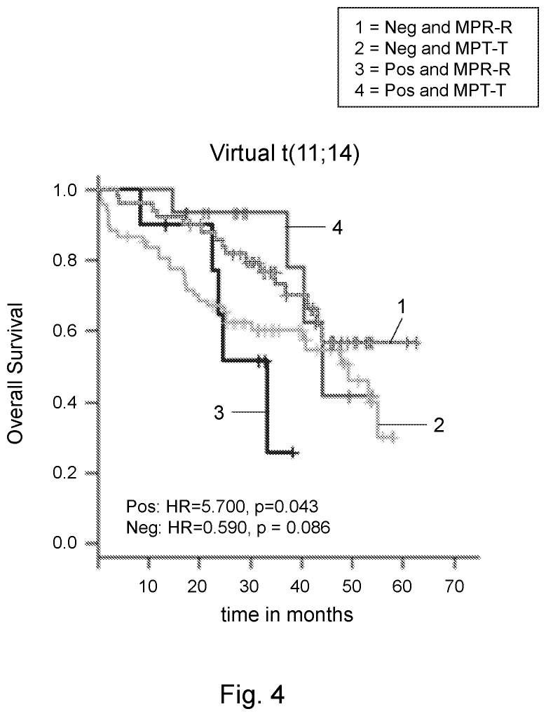

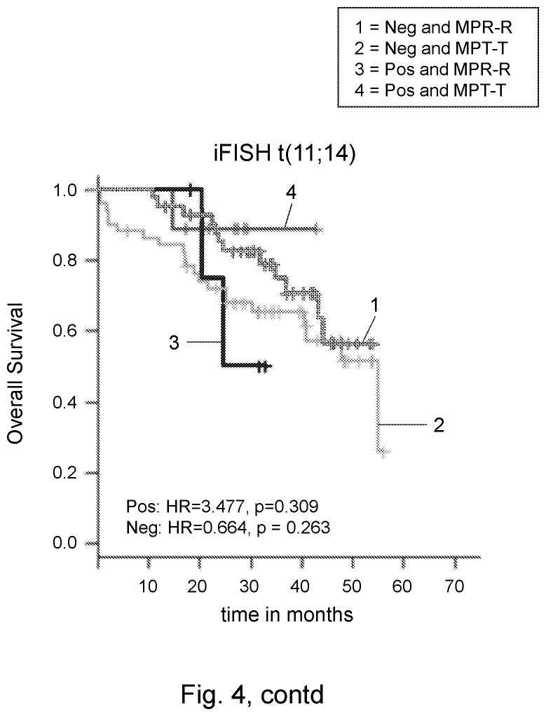

The Virtual t(11;14) marker is congruent with iFISH t(11;14), though neither is prognostic with HR=1.04, p=0.92, and HR=0.66, p=0.26, respectively, between the positive/negative groups. However, when splitting by treatment arm, there is an indication that Virtual t(11;14) positive patients have an OS advantage when treated with MPT-T over MPR-R, HR=5.7, p=0.043. At the same time, in the Virtual t(11;14) negative group there is an indication that the MPR-R treatment outperforms the MPT-T treatment at HR=0.59, p=0.086. See FIG. 4. These observations support the use of the Virtual t(11;14) and iFISH t(11;14) as a predictive marker, i.e. as a means to determine an MM patient's preferential treatment. Positive cases for the t(11;14) marker have a benefit from MPT-T treatment over MPR-R treatment, whereas the negative cases for this marker have a benefit from MPR-R treatment over MPT-T treatment.

Table 7 shows the overlap of the samples. For example, Table 7a shows that there are 9 patients which are virtual t(4;14) positive and at the same time SKY92 High Risk. Of the 143 samples, iFISH t(4:14) status was determined in 128 of the samples (Table 7b) and iFISH t(11;14) status was determined in 107 samples (fable 7c). As expected, the overlap between iFiSH and virtual translocations is high. The overlap between the t(4;14) marker and the MS cluster is also very high. Approximately half of the t(4;14) cases are also SKY92 High Risk. On the other hand, the overlap between t(11;14), and SKY92 High Risk is limited. The t(11;14) and t(4;14) translocations are mutually exclusive, which is in line with previous findings.

Table 7: Tables indicating pairwise overlap of the different markers, overlap between the same marker (diagonal entries) indicates the number of positives for that marker.

Tables 7a and 7b

128 H87 patients with iFISH t(4; 14)

| | SKY92 | Cluster | Virtual | Virtual | iFISH |
|---|---|---|---|---|---|
| | High Risk | MS | t(4; 14) | t(11; 14) | t(4; 14) |
| SKY92 High Risk | 21 | 9 | 9 | 1 | 8 |
| Cluster MS | 9 | 15 | 15 | 0 | 12 |
| Virtual t(4; 14) | 9 | 15 | 16 | 0 | 13 |
| Virtual t(11; 14) | 1 | 0 | 0 | 21 | 0 |

All 143 H87 samples

| | SKY92 | Cluster | Virtual | Virtual |
|---|---|---|---|---|
| | High Risk | MS | t(4; 14) | t(11; 14) |
| SKY92 High Risk | 22 | 9 | 9 | 2 |
| Cluster MS | 9 | 15 | 15 | 0 |
| Virtual t(4; 14) | 9 | 15 | 16 | 0 |
| Virtual t(11; 14) | 2 | 0 | 0 | 25 |

TABLE 7c
107 H87 patients with iFISH t(11; 14)

| | SKY92 | Cluster | Virtual | Virtual | iFISH |
|---|---|---|---|---|---|
| | High Risk | MS | t(4; 14) | t(11; 14) | t(11; 14) |
| SKY92 High Risk | 14 | 3 | 3 | 1 | 2 |
| Cluster MS | 3 | 8 | 8 | 0 | 0 |
| Virtual t(4; 14) | 3 | 8 | 8 | 0 | 0 |
| Virtual t(11; 14) | 1 | 0 | 0 | 20 | 13 |

TABLE 8A
Indicates pairwise overlap in terms of probesets used in the
different GEP signatures. Overlap between the same marker (diagonal
entries) indicates the number of probesets in the signature for
that marker.
Overlap Probesets in signatures

| | SKY92 | Cluster | Virtual | Virtual |
|---|---|---|---|---|
| | High Risk | MS | t(4; 14) | t(11; 14) |
| SKY92 High Risk | 92 | 2 | 1 | 0 |
| Cluster MS | 2 | 20 | 10 | 0 |
| Virtual t(4; 14) | 1 | 10 | 19 | 0 |
| Virtual t(11; 14) | 0 | 0 | 0 | 3 |

Conclusion

In conclusion, the SKY92 signature is a useful prognostic marker to identify a high-risk subgroup in the elderly population. Moreover, MM patients with SKY92 High Risk, Virtual t(4;14), iFISH t(4;14), or MS cluster characteristics have improved Overall Survival when treated with MPR-R instead of MPT-T. Conversely, MM patients with Virtual t(11;14) have an OS advantage when treated with MPT-T.

### Example 2

A further analysis was performed to demonstrate that subsets of markers from Tables 1-4 are predictive of patient response. In a specific embodiment, all single probesets and all pluralities of subsets of the 20, 19, 92, or 3 probesets from the Tables 1-4 can be employed. For each marker, the number of possible subsets was calculated using the binomial coefficient, defined as n!/((n−k)! k!). This is the number of combinations of n items taken k at a time. For example, from the list of 92 (n) probesets from SKY92, there are 4186 subsets of 2 (k). Table 8B shows the number of unique subsets that can be taken for each of the markers. For each of the markers all subsets of 1, 2, 3, and 4 probesets were evaluated. This was done using the data from the 143 patients analyzed in the HOVON-87/NMSG-18 dataset.

TABLE 8B
The amount of subsets of a specific size that can be selected from
the total number of probesets in each of the four signatures.

| | Probesets in | Subsets | Subsets | Subsets | Subsets |
|---|---|---|---|---|---|
| Signature | Signature | of 1 | of 2 | of 3 | of 4 |
| SKY92 | 92 | 92 | 4186 | 125580 | 2794155 |
| Virtual t(4; 14) | 19 | 19 | 171 | 969 | 3876 |
| MS Cluster | 20 | 20 | 190 | 1140 | 4845 |
| Virtual t(11; 14) | 3 | 3 | 3 | 1 | NA |

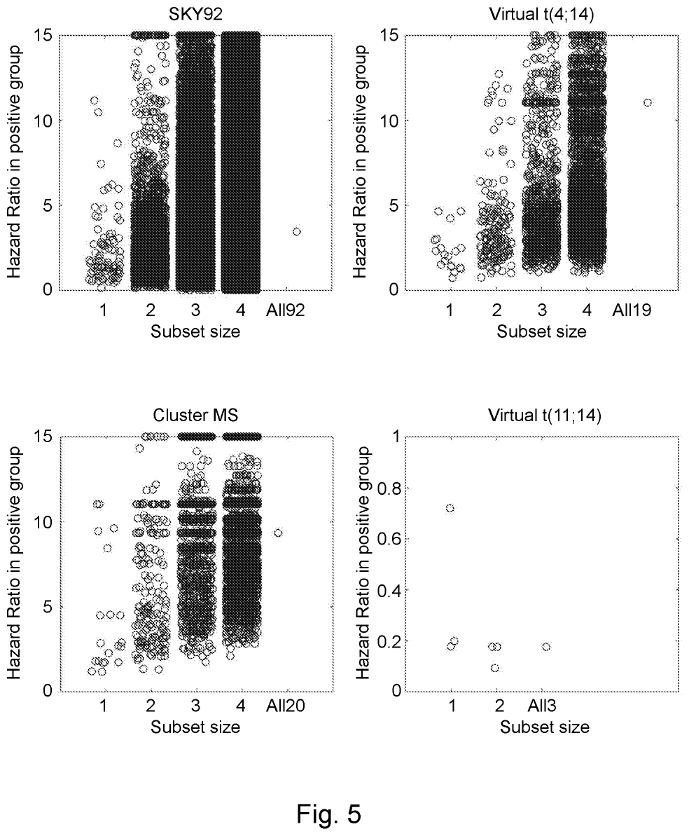

For example, for the SKY92 signature, all 4186 subsets of 2 probesets were tested. That is, when two probesets were tested, for each of the 143 samples in the HOVON-87/NMSG-18 the equation 3 was applied. In this case the summation then goes to two instead of 92. Subsequently, the 143 SKY92(x) scores were sorted, and the top 22 (=the same amount as when all 92 probesets are used) were taken as SKY92 High Risk. This ensures that the same fraction of High Risk cases are identified, as the thresholds needs to be adjusted to be applicable for the subset of probesets. Next, within those 22 SKY92 High Risk patients, a Cox Proportional Hazards model was applied using the Treatment arm as covariate, providing a Hazard Ratio g C4-/TC4sub, in the same fashion as shown in FIG. 2). All Hazard Ratios were collected, and are shown in FIG. 5.

For example, for the Virtual t(4;14) marker, all 969 subsets of 3 probesets were tested. That is, when three probesets were tested, for each of the 143 samples in the HOVON-87/NMSG-18, the equation 1 and 2 were applied. In this case the summation then goes to three instead of 19. Next, for each of the 143 samples in the HOVON-87/NMSG18 the resulting d0 and d1 were compared. Samples where d1 is smaller were classified as positive for that particular marker. Next, within those Virtual t(4;14) positive patients, a Cox Proportional Hazards model was applied using the Treatment arm as covariate, providing a Hazard Ratio (TC4-/TC4sub, in the same fashion as shown in FIG. 3, although in FIG. 3 the ratio is inverted: i.e. TC4sub/TC4-). All Hazard Ratios were collected, and are shown in FIG. 5. However, in this analysis the comparison of treatment was opposite to that shown in FIG. 3. Otherwise stated, the HRs shown in FIG. 3 could be considered as 1/HR when compared to FIG. 5.

As can be clearly seen in FIG. 5 and Tables 9 and 10, the majority of subsets (up to 100%) of each of the 4 signatures work, and indicate a benefit in terms of Overall Survival in favour of MPR-R for the SKY92, Virtual t(4;14), and MS cluster, and a benefit in terms of Overall Survival in favour of MPT-T for the Virtual t(11;14) signature.

TABLE 9
Number of tested subsets that had a Hazard Ratio
(TC4-/TC4sub) larger than 1 or smaller than 1
(i.e. in the same direction as when using all probesets).

| | Subsets | Subsets | Subsets | Subsets | All |
|---|---|---|---|---|---|
| Signature | of 1 | of 2 | of 3 | of 4 | probesets |
| | HR > 1 | | | | |
| SKY92 | 72 | 3351 | 103244 | 2332090 | 1 |
| Virtual t(4; 14) | 18 | 168 | 969 | 3876 | 1 |
| MS Cluster | 20 | 190 | 1140 | 4845 | 1 |
| | HR < 1 | | | | |
| Virtual t(11; 14) | 3 | 3 | 1 | 1 | 1 |

TABLE 10
Percentage of all tested subsets that had a Hazard Ratio (TC4-/TC4sub) larger than
1 or smaller than 1 (i.e. in the same direction as when using all probesets).

| | Subsets | Subsets | Subsets | Subsets | All |
|---|---|---|---|---|---|
| Signature | of 1 | of 2 | of 3 | of 4 | probesets |
| | HR > 1 | | | | |
| SKY92 | 78.26% | 80.05% | 82.21% | 83.46% | 100.00% |
| Virtual t(4; 14) | 94.74% | 98.25% | 100.00% | 100.00% | 100.00% |
| MS Cluster | 100.00% | 100.00% | 100.00% | 100.00% | 100.00% |
| | HR < 1 | | | | |
| Virtual t(11; 14) | 100.00% | 100.00% | 100.00% | 100.00% | 100.00% |

Table 9 shows that 72 markers from Table 1, 18 markers from Table 2 and all markers from Table 4 can, when used individually, identify patients with an improved OS for MPR-R (HR>1, indicating that MPT-T has lower OS than MPR-R). Table 11 shows an overview of the combined unique list of the 98 probesets. Table 9 also shows that all markers from Table 3 can, when used individually, identify patients with an improved OS for MPT-T (HR<1, indicating that MPT-T has higher OS than MPR-R). Table 12 shows the additional 21 probesets from Tables 1-4, which were not part of Table 11.

TABLE 11
Overview and annotation for 98 probe sets that are individually
predictive for improved OS on MPR-R when compared with MPT-T.
Gene Symbol and Gene Title information were retrieved from the Affymetrix
NetAffx website (https://www.affymetrix.com/estore/analysis/index.affx)
on Jan. 26^{th}, 2016.

| Probeset | Signature | Gene Symbol | Gene Title |
|---|---|---|---|
| 204379_s_al | Virtual | FGFR3 | fibroblast growth factor |
| | t(4; 14), | | receptor 3 |
| | Cluster MS, | | |
| | SKY92 | | |
| 211709_s_at | Virtual | CLEC11A | C-type lectin domain |
| | t(4; 14), | | family 11, member A |
| | Cluster MS | | |
| 217867_x_at | Virtual | BACE2 | beta-site APP-cleaving |
| | t(4; 14), | | enzyme 2 |
| | Cluster MS | | |
| 222258_s_at | Virtual | SH3BP4 | SH3-domain binding |
| | t(4; 14), | | protein 4 |
| | Cluster MS | | |
| 222777 s at | Virtual | WHSC1 | Wolf-Hirschhorn |
| | t(4; 14), | | syndrome candidate 1 |
| | Cluster MS | | |
| 222778_s_at | Virtual | WHSC1 | Wolf-Hirschhorn |
| | t(4; 14), | | syndrome candidate 1 |
| | Cluster MS | | |
| 223472_at | Virtual | WHSC1 | Wolf-Hirschhorn |
| | t(4; 14), | | syndrome candidate 1 |
| | Cluster MS | | |
| 223822_at | Virtual | SUSD4 | sushi domain containing |
| | t(4; 14), | | 4 |
| | Cluster MS | | |
| 227084_at | Virtual | DTNA | dystrobrevin, alpha |
| | t(4; 14), | | |
| | Cluster MS | | |
| 227692_at | Virtual | GNAI1 | guanine nucleotide |
| | t(4; 14), | | binding protein (G |
| | Cluster MS | | protein), alpha |
| | | | inhibiting activity |
| | | | polypeptide 1 |
| 238116_at | Cluster MS, | DYNLRB2 | dynein, light chain, |
| | SKY92 | | roadblock-type 2 |
| 1553105_s_at | Cluster MS | DSG2 | desmoglein 2 |
| 1557780_at | Cluster MS | — | — |
| 200701_at | SKY92 | NPC2 | Niemann-Pick disease, |
| | | | type C2 |
| 200875_s_at | SKY92 | MIR1292 /// | microRNA 1292 /// |
| | | NOP56 /// | NOP56 |
| | | SNORD110 /// | ribonucleoprotein /// |
| | | SNORD57 /// | small nucleolar RNA, |
| | | SNORD86 | C/D box 110 /// small |
| | | | nucleolar RNA, C/D box |
| | | | 57 /// small nucleolar |
| | | | RNA, C/D box 86 |
| 200933_x_at | SKY92 | RPS4X | ribosomal protein S4, X- |
| | | | linked |
| 201307_at | SKY92 | SEP11 | septin 11 |
| 201398_s_at | SKY92 | TRAM1 | translocation associated |
| | | | membrane protein 1 |
| 201555_at | SKY92 | MCM3 | minichromosome |
| | | | maintenance complex |
| | | | component 3 |
| 201795_at | SKY92 | LBR | lamin B receptor |
| 202107_s_at | SKY92 | MCM2 | minichromosome |
| | | | maintenance complex |
| | | | component 2 |
| 202532_s_at | SKY92 | DHFR | dihydrofolate reductase |
| 202542_s_at | SKY92 | AIMP1 | aminoacyl tRNA |
| | | | synthetase complex- |
| | | | interacting |
| | | | multifunctional protein |
| | | | 1 |
| 202553_s_at | SKY92 | SYF2 | SYF2 pre-mRNA- |
| | | | splicing factor |
| 202728_s_at | SKY92 | LTBP1 | latent transforming |
| | | | growth factor beta |
| | | | binding protein 1 |
| 202813_at | SKY92 | TARBP1 | TAR (HIV-1) RNA |
| | | | binding protein 1 |
| 202842_s_at | SKY92 | DNAJB9 | DnaJ (Hsp40) homolog, |
| | | | subfamily B, member 9 |
| 202884_s_at | SKY92 | PPP2R1B | protein phosphatase 2, |
| | | | regulatory subunit A, |
| | | | beta |
| 203145_at | SKY92 | SPAG5 | sperm associated |
| | | | antigen 5 |
| 204026_s_at | SKY92 | ZWINT | ZW10 interacting |
| | | | kinetochore protein |
| 204066_s_at | Cluster MS | AGAP1 | ArfGAP with GTPase |
| | | | domain, ankyrin repeat |
| | | | and PH domain 1 |
| 205046_at | SKY92 | CENPE | centromere protein E, |
| | | | 312 kDa |
| 205131_x_at | Virtual | CLEC11A | C-type lectin domain |
| | t(4; 14) | | family 11, member A |
| 205559_s_at | Cluster MS | PCSK5 | proprotein convertase |
| | | | subtilisin/kexin type 5 |
| 205830_at | Virtual | CLGN | calmegin |
| | t(4; 14) | | |
| 206204_at | SKY92 | GRB14 | growth factor receptor- |
| | | | bound protein 14 |
| 207618_s_at | SKY92 | BCS1L | BC1 (ubiquinol- |
| | | | cytochrome c reductase) |
| | | | synthesis-like |
| 208232_x_at | SKY92 | NRG1 | neuregulin 1 |
| 208667_s_at | SKY92 | ST13 | suppression of |
| | | | tumorigenicity 13 (colon |
| | | | carcinoma) (Hsp70 |
| | | | interacting protein) |
| 208732_at | SKY92 | RAB2A | RAB2A, member RAS |
| | | | oncogene family |
| 208747_s_at | SKY92 | C1S | complement component |
| | | | 1, s subcomponent |
| 208904_s_at | SKY92 | RPS28 | ribosomal protein S28 |
| 208942_s_at | SKY92 | SEC62 | SEC62 homolog (S. |
| | | | cerevisiae) |
| 208967_s_at | SKY92 | AK2 | adenylate kinase 2 |
| 209026_x_at | SKY92 | TUBB | tubulin, beta class I |
| 210334_x_at | SKY92 | BIRC5 | baculoviral IAP repeat |
| | | | containing 5 |
| 211714_x_at | SKY92 | TUBB | tubulin, beta class I |
| 211963_s_at | SKY92 | ARPC5 | actin related protein 2/3 |
| | | | complex, subunit 5, |
| | | | 16 kDa |
| 212055_at | SKY92 | TPGS2 | tubulin polyglutamylase |
| | | | complex subunit 2 |
| 212148_at | Virtual | PBX1 | pre-B-cell leukemia |
| | t(4; 14) | | homeobox 1 |
| 212151_at | Virtual | PBX1 | pre-B-cell leukemia |
| | t(4; 14) | | homeobox 1 |
| 212190_at | Cluster MS | SERPINE2 | serpin peptidase |
| | | | inhibitor, clade E |
| | | | (nexin, plasminogen |
| | | | activator inhibitor type |
| | | | 1), member 2 |
| 212282 at | SKY92 | TMEM97 | transmembrane protein |
| | | | 97 |
| 212686_at | Cluster MS | PPM1H | protein phosphatase, |
| | | | Mg2+/Mn2+ dependent, |
| | | | 1H |
| 212771_at | Cluster MS | FAM171A1 | family with sequence |
| | | | similarity 171, member |
| | | | A1 |
| 212788_x_at | SKY92 | FTL | ferritin, light |
| | | | polypeptide |
| 212813_at | Virtual | JAM3 | junctional adhesion |
| | t(4; 14) | | molecule 3 |
| 213002_at | SKY92 | MARCKS | myristoylated alanine- |
| | | | rich protein kinase C |
| | | | substrate |
| 213007_at | SKY92 | FANCI | Fanconi anemia, |
| | | | complementation group |
| | | | I |
| 213350_at | SKY92 | RPS11 | ribosomal protein S11 |
| 214156_at | Cluster MS | MYRIP | myosin VIIA and Rab |
| | | | interacting protein |
| 215177_s_at | SKY92 | ITGA6 | integrin, alpha 6 |
| 215181_at | SKY92 | CDH22 | cadherin 22, type 2 |
| 217548_at | SKY92 | ARPIN | actin-related protein 2/3 |
| | | | complex inhibitor |
| 217728_at | SKY92 | S100A6 | S100 calcium binding |
| | | | protein A6 |
| 217732_s_at | SKY92 | ITM2B | integral membrane |
| | | | protein 2B |
| 217824_at | SKY92 | UBE2J1 | ubiquitin-conjugating |
| | | | enzyme E2, J1 |
| 217852_s_at | SKY92 | ARL8B | ADP-ribosylation factor- |
| | | | like 8B |
| 217901_at | Cluster MS | DSG2 | desmoglein 2 |
| 218365_s_at | SKY92 | DARS2 | aspartyl-tRNA |
| | | | synthetase 2, |
| | | | mitochondrial |
| 219510_at | SKY92 | POLQ | polymerase (DNA |
| | | | directed), theta |
| 219550_at | SKY92 | ROBO3 | roundabout, axon |
| | | | guidance receptor, |
| | | | homolog 3 (Drosophila) |
| 221041_s_at | SKY92 | SLC17A5 | solute carrier family 17 |
| | | | (acidic sugar |
| | | | transporter), member 5 |
| 221261_x_at | Virtual | MAGED4 /// | melanoma antigen |
| | t(4; 14) | MAGED4B /// | family D, 4 /// melanoma |
| | | SNORA11D /// | antigen family D, 4B /// |
| | | SNORA11E | small nucleolar RNA, |
| | | | H/ACAbox 11D /// small |
| | | | nucleolar RNA, H/ACA |
| | | | box 11E |
| 221606_s_at | SKY92 | HMGN5 | high mobility group |
| | | | nucleosome binding |
| | | | domain 5 |
| 221755_at | SKY92 | EHBP1L1 | EH domain binding |
| | | | protein 1-like 1 |
| 222154_s_at | SKY92 | SPATS2L | spermatogenesis |
| | | | associated, serine-rich |
| | | | 2-like |
| 222680_s_at | SKY92 | DTL | denticleless E3 |
| | | | ubiquitin protein ligase |
| | | | homolog (Drosophila) |
| 222713_s_at | SKY92 | FANCF | Fanconi anemia, |
| | | | complementation group |
| | | | F |
| 224009_x_at | SKY92 | DHRS9 | dehydrogenase/reductase |
| | | | (SDR family) member |
| | | | 9 |
| 225366_at | SKY92 | PGM2 | phosphoglucomutase 2 |
| 225601_at | SKY92 | HMGB3 | high mobility group box |
| | | | 3 |
| 226217_at | SKY92 | SLC30A7 | solute carrier family 30 |
| | | | (zinc transporter), |
| | | | member 7 |
| 226218_at | SKY92 | IL7R | interleukin 7 receptor |
| 226742_at | SKY92 | SAR1B | secretion associated, |
| | | | Ras related GTPase 1B |
| 227290_at | Virtual | CDYL2 | chromodomain protein, |
| | t(4; 14) | | Y-like 2 |
| 227434_at | Virtual | WBSCR17 | Williams-Beuren |
| | t(4; 14) | | syndrome chromosome |
| | | | region 17 |
| 230034_x_at | SKY92 | MRPL41 | mitochondrial ribosomal |
| | | | protein L41 |
| 231210_at | SKY92 | C11orf85 | chromosome 11 open |
| | | | reading frame 85 |
| 231738_at | SKY92 | PCDHB7 | protocadherin beta 7 |
| 231989_s_at | SKY92 | LOC101060604 /// | putative L-type amino |
| | | LOC101929910 /// | acid transporter 1-like |
| | | LOC102725125 /// | protein IMAA-like /// |
| | | LOC613037 /// | nuclear pore complex- |
| | | NPIPA5 /// | interacting protein |
| | | NPIPB3 /// | family member B4-like /// |
| | | NPIPB4 /// | serine/threonine- |
| | | NPIPB5 /// | protein kinase SMG1- |
| | | SLC7A5P1 /// | like /// nuclear pore |
| | | SMG1P1 /// | complex interacting |
| | | SMG1P3 | protein pseudogene /// |
| | | | nuclear pore complex |
| | | | interacting protein |
| | | | family, member A5 /// |
| | | | nuclear pore complex |
| | | | interacting protein |
| | | | family, member B3 /// |
| | | | nuclear pore complex |
| | | | interacting protein |
| | | | family, member B4 /// |
| | | | nuclear pore complex |
| | | | interacting protein |
| | | | family, member B5 /// |
| | | | solute carrier family 7 |
| | | | (amino acid transporter |
| | | | light chain, L system), |
| | | | member 5 pseudogene 1 /// |
| | | | SMG1 pseudogene 1 /// |
| | | | SMG1 pseudogene 3 |
| 233399_x_at | SKY92 | ZNF252P | zinc finger protein 252, |
| | | | pseudogene |
| 233437_at | SKY92 | GABRA4 | gamma-aminobutyric |
| | | | acid (GABA) A receptor, |
| | | | alpha 4 |
| 238662_at | SKY92 | DPH6 | diphthamine |
| | | | biosynthesis 6 |
| 239054_at | SKY92 | SFMBT1 | Scm-like with four mbt |
| | | | domains 1 |
| 243018_at | SKY92 | RP11-1L12.3 | — |
| 38158_at | SKY92 | ESPL1 | extra spindle pole |
| | | | bodies homolog 1 |
| | | | (S. cerevisiae) |
| AFFX- | SKY92 | STAT1 | signal transducer and |
| HUMISGF3A/M97935_MA_at | | | activator of |
| | | | transcription 1, 91 kDa |

TABLE 12
Gene Symbol and Gene Title information were retrieved from the Affymetrix NetAffx
website (https://www.affymetrix.com/estore/analysis/index.affx) on Jan. 26^{th}, 2016.

| Probeset | Signature | Gene Symbol | Gene Title |
|---|---|---|---|
| 200775_s_at | SKY92 | HNRNPK | heterogeneous nuclear |
| | | | ribonucleoprotein K |
| 201102_s_at | SKY92 | PFKL | phosphofructokinase, liver |
| 201292_at | SKY92 | TOP2A | topoisomerase (DNA) II alpha |
| | | | 170 kDa |
| 201930_at | SKY92 | MCM6 | minichromosome maintenance |
| | | | complex component 6 |
| 202322_s_at | SKY92 | GGPS1 | geranylgeranyl diphosphate |
| | | | synthase 1 |
| 209683_at | SKY92 | FAM49A | family with sequence similarity |
| | | | 49, member A |
| 214150_x_at | SKY92 | ATP6V0E1 | ATPase, H+ transporting, |
| | | | lysosomal 9 kDa, V0 subunit e1 |
| 214482_at | SKY92 | ZBTB25 | zinc finger and BTB domain |
| | | | containing 25 |
| 214612_x_at | SKY92 | MAGEA6 | melanoma antigen family A, 6 |
| 216473_x_at | SKY92 | DBET /// DUX4 /// | D4Z4 binding element transcript |
| | | DUX4L1 /// DUX4L2 /// | (non-protein coding) /// double |
| | | DUX4L24 /// | homeobox 4 /// double homeobox 4 |
| | | DUX4L3 /// DUX4L4 /// | like 1 /// double homeobox 4 like 2 /// |
| | | DUX4L5 /// | double homeobox 4 like 24 /// |
| | | DUX4L6 /// DUX4L7 /// | double homeobox 4 like 3 /// |
| | | DUX4L8 /// | double homeobox 4 like 4 /// |
| | | LOC100288289 /// | double homeobox 4 like 5 /// |
| | | LOC100291626 /// | double homeobox 4 like 6 /// |
| | | LOC652301 | double homeobox 4 like 7 /// |
| | | | double homeobox 4 like 8 /// |
| | | | double homeobox protein 4-like |
| | | | protein 2-like /// double homeobox |
| | | | protein 4-like /// double homeobox |
| | | | protein 4-like protein 4-like |
| 218355_at | SKY92 | KIF4A | kinesin family member 4A |
| 218662_s_at | SKY92 | NCAPG | non-SMC condensin I complex, |
| | | | subunit G |
| 220351_at | SKY92 | ACKR4 | atypical chemokine receptor 4 |
| 221677_s_at | SKY92 | DONSON | downstream neighbor of SON |
| 221826_at | SKY92 | ANGEL2 | angel homolog 2 (Drosophila) |
| 223313_s_at | Virtual | MAGED4 /// | melanoma antigen family D, 4 /// |
| | t(4; 14) | MAGED4B /// | melanoma antigen family D, 4B /// |
| | | SNORA11D/// | small nucleolar RNA, H/ACA box |
| | | SNORA11E | 11D /// small nucleolar RNA, |
| | | | H/ACA box 11E |
| 223381_at | SKY92 | NUF2 | NUF2, NDC80 kinetochore |
| | | | complex component |
| 223811_s_at | SKY92 | GET4 /// SUN1 | golgi to ER traffic protein 4 |
| | | | homolog (S. cerevisiae) /// Sad1 |
| | | | and UNC84 domain containing 1 |
| 228416_at | SKY92 | ACVR2A | activin A receptor, type IIA |
| 238780_s_at | SKY92 | KCNJ5 | potassium inwardly-rectifying |
| | | | channel, subfamily J, member 5 |
| 242180_at | SKY92 | TSPAN16 | tetraspanin 16 |

TABLE 13
Markers present in both Table 1 and Table 11. Exemplary beta values (i.e., weights)
and thresholds are provided for each probeset. The thresholds were determined such
that each individual probeset classifies an individual as disclosed herein.

| Probesets | Beta | Gene Symbol | Threshold |
|---|---|---|---|
| 200701_at | −0.0210 | NPC2 | 0.0190 |
| 200775_s_at | 0.0163 | HNRNPK /// MIR7-1 | 0.0152 |
| 200875_s_at | 0.0437 | MIR1292 /// | 0.0385 |
| | | NOP56 /// | |
| | | SNORD110 /// | |
| | | SNORD57 /// SNORD86 | |
| 200933_x_at | −0.0323 | RPS4X | 0.0245 |
| 201102_s_at | 0.0349 | PFKL | 0.0449 |
| 201292_at | −0.0372 | TOP2A | 0.0310 |
| 201307_at | 0.0165 | SEPT11 | 0.0181 |
| 201398_s_at | −0.0254 | TRAM1 | 0.0263 |
| 201555_at | −0.0052 | MCM3 | 0.0037 |
| 201795_at | 0.0067 | LBR | 0.0069 |
| 201930_at | −0.0090 | MCM6 | 0.0091 |
| 202107_s_at | 0.0225 | MCM2 | 0.0266 |
| 202322_s_at | 0.0129 | GGPS1 | 0.0153 |
| 202532_s_at | −0.0006 | DHFR | 0.0005 |
| 202542_s_at | 0.0870 | AIMP1 | 0.0945 |
| 202553_s_at | 0.0054 | SYF2 | 0.0054 |
| 202728_s_at | −0.1105 | LTBP1 | 0.0998 |
| 202813_at | 0.0548 | TARBP1 | 0.0472 |
| 202842_s_at | −0.0626 | DNAJB9 | 0.0776 |
| 202884_s_at | 0.0714 | PPP2R1B | 0.0544 |
| 203145_at | −0.0002 | SPAG5 | 0.0002 |
| 204026_s_at | 0.0046 | ZWINT | 0.0049 |
| 204379_s_at | 0.0594 | FGFR3 | 0.0052 |
| 205046_at | 0.0087 | CENPE | 0.0105 |
| 206204_at | 0.0477 | GRB14 | 0.0606 |
| 207618_s_at | 0.0746 | BCS1L | 0.0660 |
| 208232_x_at | −0.0493 | NRG1 | 0.0801 |
| 208667_s_at | −0.0390 | ST13 | 0.0395 |
| 208732_at | −0.0618 | RAB2A | 0.0698 |
| 208747_s_at | −0.0874 | C1S | 0.0882 |
| 208904_s_at | −0.0334 | RPS28 | 0.0247 |
| 208942_s_at | −0.0997 | SEC62 | 0.0935 |
| 208967_s_at | 0.0113 | AK2 | 0.0087 |
| 209026_x_at | 0.0255 | TUBB | 0.0316 |
| 209683_at | −0.0561 | FAM49A | 0.0293 |
| 210334_x_at | 0.0175 | BIRC5 | 0.0193 |
| 211714_x_at | 0.0221 | TUBB | 0.0287 |
| 211963_s_at | 0.0303 | ARPC5 | 0.0334 |
| 212055_at | 0.0384 | TPGS2 | 0.0352 |
| 212282_at | 0.0530 | TMEM97 | 0.0515 |
| 212788_x_at | −0.0164 | FTL | 0.0131 |
| 213002_at | −0.0418 | MARCKS | 0.0385 |
| 213007_at | −0.0106 | FANCI | 0.0099 |
| 213350_at | 0.0056 | RPS11 | 0.0087 |
| 214150_x_at | −0.0349 | ATP6V0E1 | 0.0243 |
| 214482_at | 0.0861 | ZBTB25 | 0.0834 |
| 214612_x_at | 0.0496 | MAGEA6 | 0.0611 |
| 215177_s_at | −0.0768 | ITGA6 | 0.0835 |
| 215181_at | −0.0342 | CDH22 | 0.0380 |
| 216473_x_at | −0.0576 | DUX2 /// DUX4 /// DUX4L2 /// | 0.0664 |
| | | DUX4L3 /// DUX4L4 /// | |
| | | DUX4L5 /// | |
| | | DUX4L6 /// DUX4L7 /// | |
| | | LOC100288627/// | |
| | | LOC100288657 /// | |
| | | LOC652119 | |
| 217548_at | −0.0423 | LOC100129502 | 0.0460 |
| 217728_at | 0.0773 | S100A6 | 0.0740 |
| 217732_s_at | −0.0252 | ITM2B | 0.0297 |
| 217824_at | −0.0041 | UBE2J1 | 0.0035 |
| 217852_s_at | 0.0008 | ARL8B | 0.0007 |
| 218355_at | 0.0116 | KIF4A | 0.0126 |
| 218365_s_at | 0.0035 | DARS2 | 0.0028 |
| 218662_s_at | −0.0176 | NCAPG | 0.0213 |
| 219510_at | −0.0097 | POLQ | 0.0093 |
| 219550_at | 0.0559 | ROBO3 | 0.0522 |
| 220351_at | 0.0420 | CCRL1 | 0.0383 |
| 221041_s_at | −0.0520 | SLC17A5 | 0.0369 |
| 221606_s_at | 0.0208 | HMGN5 | 0.0163 |
| 221677_s_at | 0.0126 | DONSON | 0.0146 |
| 221755_at | 0.0396 | EHBP1L1 | 0.0317 |
| 221826_at | 0.0200 | ANGEL2 | 0.0147 |
| 222154_s_at | 0.0154 | SPATS2L | 0.0148 |
| 222680_s_at | 0.0205 | DTL | 0.0213 |
| 222713_s_at | 0.0278 | FANCF | 0.0239 |
| 223381_at | −0.0070 | NUF2 | 0.0106 |
| 223811_s_at | 0.0556 | GET4 /// SUN1 | 0.0562 |
| 224009_x_at | −0.0520 | DHRS9 | 0.0583 |
| 225366_at | 0.0140 | PGM2 | 0.0139 |
| 225601_at | 0.0750 | HMGB3 | 0.0659 |
| 226217_at | −0.0319 | SLC30A7 | 0.0229 |
| 226218_at | −0.0644 | IL7R | 0.0675 |
| 226742_at | −0.0345 | SAR1B | 0.0312 |
| 228416_at | −0.0778 | ACVR2A | 0.1187 |
| 230034_x_at | −0.0330 | MRPL41 | 0.0257 |
| 231210_at | 0.0093 | C11orf85 | 0.0093 |
| 231738_at | 0.0686 | PCDHB7 | 0.0714 |
| 231989_s_at | 0.0730 | 61E3.4 /// LOC100132247 /// | 0.0681 |
| | | LOC100271836 /// | |
| | | LOC100652992 /// | |
| | | LOC613037 /// | |
| | | LOC728888 /// NPIPL3 /// | |
| | | SLC7A5P1 ///SMG1P1 | |
| 233399_x_at | −0.0184 | TMED10P1 ///ZNF252 | 0.0182 |
| 233437_at | 0.0446 | GABRA4 | 0.0493 |
| 238116_at | 0.0661 | DYNLRB2 | 0.0811 |
| 238662_at | 0.0490 | ATPBD4 | 0.0452 |
| 238780_s_at | −0.0529 | — | 0.0551 |
| 239054_at | −0.1088 | SFMBT1 | 0.0904 |
| 242180_at | −0.0585 | TSPAN16 | 0.0546 |
| 243018_at | 0.0407 | — | 0.0484 |
| 38158_at | 0.0423 | ESPL1 | 0.0424 |
| AFFX- | 0.0525 | STAT1 /// STAT1 | 0.0354 |
| HUMISGF3A/M97935_MA_at | | | |

### REFERENCES

1. Brenner H, Gondos A, Pulte D. Recent major improvement in long-term survival of younger patients with multiple myeloma. Blood. 2008; 111:2521-2526.
2. Ocio E M, Richardson P G, Rajkumar S V, et al. New drugs and novel mechanisms of action in multiple myeloma in 2013: a report from the International Myeloma Working Group (IMWG). Leukemia. 2014; 28(3):525-42
3. Kumar S K, Rajkumar S V, Dispenzieri A, et al. Improved survival in multiple myeloma and the impact of novel therapies. Blood 2008; 111:2516-20
4. Sonneveld P, Schmidt-Wolf I G H, van der Holt B, et al. Bortezomib Induction and Maintenance Treatment in Patients With Newly Diagnosed Multiple Myeloma: Results of the Randomized Phase III HOVON-65/GMMG-HD4 Trial. JCO. 2012; 30(24):2946-2955
5 Sonneveld P, Goldschmidt H, Rosinol L, et al. Bortezomib-Based Versus Nonbortezomib-Based Induction Treatment Before Autologous Stem-Cell Transplantation in Patients With Previously Untreated Multiple Myeloma: A Meta-Analysis of Phase III Randomized, Controlled Trials. JCO. 2013; 31(26):3279-3287
6. Broyl A, Hose D, Lokhorst H, et al. Gene expression profiling for molecular classification of multiple myeloma in newly diagnosed patients. Blood. 2010; 116:2543-2553.
7. Zhou Y, Zhang Q, Stephens O, et al. Prediction of cytogenetic abnormalities with gene expression profiles. Blood. 2012;119:e148-e150
8. Van Vliet M, Kuiper R, Broijl A, et al. An assay for simultaneous diagnosis of t(4;14), t(11;14), 414;16)/t(14;20), del1p, add1q, del13q, del17p, MS/MF expression clusters, and the SKY-92 high risk signature in multiple myeloma patients. EHA abstract P234. 2013.
9. Avet-Loiseau H, Leleu X, Roussel M, et al. Bortezomib Plus Dexamethasone Induction Improves Outcome of Patients With 44;14) Myeloma but Not Outcome of Patients With del(17p). JCO. 2010; 28(30):4630-4634
10. Goldschmidt H, Neben K, Bertsch U, et al. Bortezomib-Based Induction Therapy Followed by Autologous Stem Cell Transplantation and Maintenance Therapy with Bortezomib Improves Outcome In Myeloma Patients with Gain 1q21 and 44;14)—a Subgroup Analysis of the HOVON-65/GMMG-HD4 Trial. ASH. 2010; Abstract 305
11. Ito T, Ando H, Suzuki T, et al. Identification of a primary target of thalidomide teratogenicity. Science. 2010; 327(5971): 1345-1350
12. Broyl A, Kuiper R, van Duin M, et al. High cereblon expression is associated with better survival in patients with newly diagnosed multiple myeloma treated with thalidomide maintenance. Blood. 2013; 121:624-627
13. Schuster S R, Kortuem K M, Zhu Y X, et al. The clinical significance of cereblon expression in multiple myeloma. Leukemia Research. 2014; 38:23-28
14. Kuiper R, Broyl A, de Knegt Y, et al. A gene expression signature for high-risk multiple myeloma. Leukemia. 2012; 26:2406-2413.
15. Zhan F, Huang Y, Colla S, et al. The molecular classification of multiple myeloma. Neoplasia. 2006; 108:2020-2028
16. Van Vliet M H, Kuiper R, Broijl A, et al. Combining FISH and GEP Signatures in Multiple Myeloma; Towards a More Robust and Meaningful High Risk Definition. EHA abstract #580. 2013.
17. Kuiper R, Van Duin M, Van Vliet M H, et al. Prediction of high- and low-risk multiple myeloma based on gene expression and the International Staging System. Blood, 2015; Accepted, First Edition Online
18. Chng W J, Dispenzieri, A, Chim, C S, et al. IMWG consensus on risk stratification in multiple myeloma. Leukemia. 2014; 28(2):269-77
19. Van Duin M, Kuiper R, Van Vliet et al. Validation of the EMC92/SKY92 Signature in HOVON-87/Nmsg-18: Gene Expression Based Prognostication Is Applicable in Elderly Patients with Newly Diagnosed Multiple Myeloma. ASH 57, Poster presentation (#2967), Orlando, Fla., USA, Dec. 5-8, 2015
20. van Vliet M H, Kuiper R, Dumee B, et al. Single Sample Application of the EMC92/SKY92 Signature Using the MMprofiler. ASH 56, Poster presentation (#2026), San Francisco, USA, Dec. 6-9, 2014
21. van Vliet M H, Kuiper R, Broijl A, et al. Proteasome Inhibitor Treatment Response Can Be Predicted By Gene Expression Profiling In Multiple Myeloma. EHA 19, Haematologica 2014. abstract n. 51286
22. Dabney, A R, Classification of microarrays to nearest centroids. Bioinformatics. 2005 21(22):4148-4154.
23. Fischer, E S, Bohm, K, ydeard, J R, et al. Structure of the DDB1-CRBN E3 ubiquitin ligase in complex with thalidomide. Nature 2014; 512:49-53

1-17. (canceled)

18. A method for classifying an individual with multiple myeloma based on the likelihood of response to treatment with an immunomodulatory drug (IMiD), the method comprising gene expression profiling, wherein said individual is classified as
i) a likely responder to thalidomide or an analog thereof which is not substituted with NH2 or CH3 at the C4 of the phthaloyl ring and a likely responder to a thalidomide analog which is substituted with NH2 or CH3 at the C4 of the phthaloyl ring,
ii) a likely responder to a thalidomide analog which is substituted with NH2 or CH3 at the C4 of the phthaloyl ring and a likely non-responder to thalidomide or an analog thereof which is not substituted with NH2 or CH3 at the C4 of the phthaloyl ring, or
iii) a likely non-responder to a thalidomide analog which is substituted with NH2 or CH3 at the C4 of the phthaloyl ring and a likely responder to thalidomide or an analog thereof which is not substituted with NH2 or CH3 at the C4 of the phthaloyl ring; the method comprising:
a) determining in a sample from said individual the level of expression of each marker listed in Table 1;
b) determining in a sample from said individual the level of expression of at least one marker selected from Table 11;
c) determining in a sample from said individual the presence of the t(4;14) translocation using gene expression profiling;
d) determining in a sample from said individual the level of expression of at least one marker in Table 3; and/or
e) determining in a sample from said individual the presence of the t(11;14) translocation using gene expression profiling;
wherein the individual is classified based on at least one of steps a), b), c), d) and e).

19. The method of claim 18, wherein the method comprises
a) determining in a sample from said individual the level of expression of at least one markers selected from Table 11 and/or
b) determining in a sample from said individual the presence of the t(4;14) translocation; and
c) determining in a sample from said individual the level of expression of at least one marker in Table 3 and/or d) determining in a sample from said individual the presence of the t(11;14) translocation;
wherein the individual is classified based on steps a) and/or b) and on steps c) and/or d).

20. The method of claim 18 comprising determining the level of expression of the markers from Table 2, and/or the markers from Table 4.

21. The method of claim 18, wherein the level of marker expression is determined by detection of RNA.

22. The method of claim 18, wherein the thalidomide analog which is substituted with NH₂ or CH₃ at the C4 of the phthaloyl ring is lenalidomide or pomalidomide.

23. The method of claim 18, wherein the sample comprises plasma cells.

24. A method for treating an individual for multiple myeloma comprising
a) determining in a sample from said individual the level of expression of each marker listed in Table 1;
b) determining in a sample from said individual the level of expression of at least one marker selected from Table 11;
c) determining in a sample from said individual the presence of the t(4;14) translocation using gene expression profiling;
d) determining in a sample from said individual the level of expression of at least one marker in Table 3; and/or
e) determining in a sample from said individual the presence of the t(11;14) translocation using gene expression profiling;
determining based on steps a), b), c), d) and/or e) a treatment of the individual, and treating said individual accordingly.

25. The method of claim 24, wherein the method comprises
a) determining in a sample from said individual the level of expression of at least one markers selected from Table 11 and/or
b) determining in a sample from said individual the presence of the t(4;14) translocation; and
c) determining in a sample from said individual the level of expression of at least one marker in Table 3 and/or
d) determining in a sample from said individual the presence of the t(11;14) translocation;
wherein the individual is classified based on steps a) and/or b) and on steps c) and/or d).

26. The method of claim 24 comprising determining the level of expression of the markers from Table 2, and/or the markers from Table 4.

27. The method of claim 24, wherein the level of marker expression is determined by detection of RNA.

28. The method of claim 24, wherein the thalidomide analog which is substituted with NH₂ or CH₃ at the C4 of the phthaloyl ring is lenalidomide or pomalidomide.

29. The method of claim 24 wherein the sample comprises plasma cells.

30. The method of claim 24, wherein said individual is treated with thalidomide or an analog thereof which is not substituted with NH₂ or CH₃ at the C4 of the phthaloyl ring.

31. The method of claim 24, wherein said individual is treated with a thalidomide analog which is substituted with NH₂ or CH₃ at the C4 of the phthaloyl ring.

32. A method for classifying an individual with multiple myeloma based on the likelihood of response to treatment with an immunomodulatory drug (IMiD), wherein said individual is classified as
i) a likely responder to thalidomide or an analog thereof which is not substituted with NH₂ or CH₃ at the C4 of the phthaloyl ring and a likely responder to a thalidomide analog which is substituted with NH₂ or CH₃ at the C4 of the phthaloyl ring,
ii) a likely responder to a thalidomide analog which is substituted with NH₂ or CH₃ at the C4 of the phthaloyl ring and a likely non-responder to thalidomide or an analog thereof which is not substituted with NH₂ or CH₃ at the C4 of the phthaloyl ring, or
iii) a likely non-responder to a thalidomide analog which is substituted with NH₂ or CH₃ at the C4 of the phthaloyl ring and a likely responder to thalidomide or an analog thereof which is not substituted with NH₂ or CH₃ at the C4 of the phthaloyl ring; the method comprising:
a) determining in a sample from said individual the level of expression of at least one marker selected from Table 11;
b) determining in a sample from said individual the presence of the t(4;14) translocation;
c) determining in a sample from said individual the level of expression of at least one marker in Table 3; and/or
d) determining in a sample from said individual the presence of the t(11;14) translocation; wherein the individual is classified based on at least one of steps a), b), c), and d).

33. The method of claim 32, wherein the presence of the t(4;14) translocation and/or the t(11;14) translocation is determined using fluorescence in situ hybridization (FISH).
